SEQUENCE LISTING (18 sequences)

SEQ ID NO: 1 - protein, 482 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKAKPTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - DNA, 1449 bp, Homo sapiens
atgtacagaatgcagctgctgtcctgcatcgccctgagcctggccctggtgaccaatagcgaggtgcaactcctggagtccggcggaggcctggtccaacctggaggaagcctgaggctgagctgtgccgccagcggcttcaccttttccagcttctccatgagctgggtgagacaggcccccggcaaaggcctggagtgggtgtccagcatctccggcagctccggcaccacctactatgctgattccgtgaagggcaggttcaccatctccagggacaacagcaagaacacactgtacctccaaatgaactccctgagggccgaagacaccgccgtgtactactgcgccaagccctttccctatttcgactattggggccagggcacactggtcaccgtgagctccggcgatggaagcagcggaggaagcggaggcgctagcgaaatcgtgctgacccagagccctggcacactgtccctgagccctggagaaagagccaccctgagctgtagggcctcccagagcgtgagcagcagcttcctggcctggtaccaacagaagcccggacaggcccccaggctgctgatctactatgcctcctccagggccacaggcatccccgacaggttctccggctccggttctggcaccgattttaccctgaccatctccaggctggagcccgaagacttcgccgtgtattactgccagcagaccggacgtattcctcctacctttggccagggcaccaaggtggagatcaaagccaagcccaccaccacacctgcccctagaccccctacacctgcccccacaatcgcttcccagcctctgtccctgaggcctgaggcttgtaggcctgccgctggaggagctgtgcacaccagaggcctcgacttcgcctgcgacatctatatctgggctcctctggccggcacctgtggagtcctcctgctgagcctggtgatcacactgtacaagagaggcaggaagaagctgctgtacatcttcaagcaacccttcatgaggcctgtgcagaccacccaggaagaagatggctgcagctgcaggttccctgaggaagaagagggcggatgcgagctgagagtgaagttcagcaggtccgccgatgcccctgcctatcagcagggccagaaccagctgtacaacgaactcaacctgggcaggagggaggagtacgacgtcctcgacaagaggagaggcagggaccccgagatgggaggcaagcctcagaggaggaagaaccctcaagagggactgtacaacgagctgcagaaggacaagatggccgaggcctactccgagatcggcatgaagggcgagagaagaagaggcaagggccatgatggcctctaccagggcctgagcaccgccaccaaggacacatacgatgccctgcatatgcaggccctcccccctaggtga

SEQ ID NO: 3 - protein, 481 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKAKPTTTPAPRPPTPAPTIASQPLSLRPEACRPAAGGAVHTRGLDFACDIYIWAPLAGTCGVLLLSLVITLYRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 4 - protein, 522 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKAKPTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELCVRCRHRRRQAERMSQIKRLLSEKKTCQCPHRFQKTCSPIRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 514 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKAKPTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELLYCNHRNRRRVCKCPRPVVKSGDKPSLSARYVRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 6 - protein, 514 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKAKPTTTPAPRPPTPAPTIASQPLSLRPEACRPAAGGAVHTRGLDFACDIYIWAPLAGTCGVLLLSLVITLYLYCNHRNRRRVCKCPRPVVKSGDKPSLSARYVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 7 - protein, 514 aa, Homo sapiens; identical to SEQ ID NO: 6

SEQ ID NO: 8 - protein, 408 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKAKPTTTPAPRPPTPAPTIASQPLSLRPEACRPAAGGAVHTRGLDFACDIYIWAPLAGTCGVLLLSLVITLYCVRCRHRRRQAERMSQIKRLLSEKKTCQCPHRFQKTCSPIRSKRSRLLHSDYMNMTPRRPGPTRKHYQPYAPPRDFAAYRS

SEQ ID NO: 9 - protein, 339 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 10 - protein, 352 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 11 - protein, 438 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKAKPTTTPAPRPPTPAPTIASQPLSLRPECARPAAGGAVHQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - protein, 440 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKAKPTTTPAPRPPTPAPTIASQPLSLRPEACRPAAGGAVHTRGLDFACDIYIWAPLAGTCGVLLLSLVITLYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - protein, 446 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGRASGDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 14 - protein, 451 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 15 - protein, 456 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 16 - protein, 497 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIRSKRSRLLHSDYMNMTPRRPGPTRKHYQPYAPPRDFAAYRS

SEQ ID NO: 17 - protein, 498 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIKRGRKKLLYIFKQPFMRPVQTTQEEDGCSCRFPEEEEGGCEL

SEQ ID NO: 18 - protein, 512 aa, Homo sapiens
MYRMQLLSCIALSLALVTNSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGRASGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHHH

(19) United States
(12) Patent Application Publication
(10) Pub. No.: US 2023/0364236 A1
(43) Pub. Date: Nov. 16, 2023

(54) FIBRONECTIN EXTRA DOMAIN B (EDB) -SPECIFIC CAR-T FOR CANCER

(71) Applicant: JIANGSU CELL TECH MEDICAL RESEARCH INSTITUTE CO., LTD., Nanjing (CN)

(72) Inventors: Meijia Yang, Scituate, MA (US); Zhijie Zhang, Guizhou (CN); Shiying Liao, Fujian (CN); Yinghua Miao, Jiangsu (CN); Hongping Yin, Jiangsu (CN)

(21) Appl. No.: 18/028,698

(22) PCT Filed: Sep. 27, 2021
(86) PCT No.: PCT/CN2021/120909

§ 371 Date: Mar. 27, 2023

(30) Foreign Application Priority Data
Sep. 27, 2020 (WO) PCT/CN2020/118184

(51) Int. Cl. A61K 39/00; C07K 16/18; C07K 14/705; C12N 5/0783; A61P 9/00; A61K 45/06; C07K 14/725; A61P 35/00

(52) U.S. Cl. CPC A61K 39/4631; C07K 16/18; A61K 39/4611; C07K 14/70517; C07K 14/70521; C12N 5/0638; A61P 9/00; A61K 45/06; C07K 14/7051; A61K 39/4643; A61P 35/00; C07K 2317/622; C07K 2319/03; C07K 2319/33

(57) ABSTRACT

Provided is chimeric antigen receptor (CAR) specific for the extra domain B (EDB) of fibronectin, which can be used in immune cells (such as T cells and NK cells) for treating diseases such as cancer and inflammatory disease.

### BACKGROUND OF THE INVENTION

Chimeric antigen receptors (CARs) are engineered receptors that combine both antigen-binding and immune cell (e.g., T-cell) activating functions into a single receptor, which then confers immune cells having such engineered receptors new ability to target a specific protein.

CARs have recently been used in therapies in cancer therapy, based on modified T cells with newly acquired ability to recognize cancer antigens on cancer cells in order to more effectively target and destroy them. Typically, autologous T cells are harvested from a patient in need of CAR T therapy, before engineered CARs are introduced into the isolated T cells ex vivo, before infusing the resulting CAR-T cells back into the patient to attack the tumors bearing antigen recognized by CARs.

CAR-T cells can be either derived from T cells in a patient's own blood (autologous), or more recently derived from the T cells of another healthy donor (allogeneic). For safety, CAR-T cells are preferably engineered to be specific to an antigen expressed on a tumor that is not expressed on healthy cells. Once CAR-T cells are infused into a patient, they act as a “living drug” against cancer cells, in that the CAR-T cells bind to the cancer antigen and become activated, leading to their proliferation and cytotoxicity against the cancer cells.

CAR-T cells can destroy cancer cells through several mechanisms, including extensive stimulated cell proliferation, increasing the degree to which they are toxic to other living cells (cytotoxicity) and by causing the increased secretion of factors that can affect other cells such as cytokines, interleukins and growth factors.

In recent years, CAR-T cell immunotherapy has achieved highly effective results in treating hematological malignancies. Despite significant progress, however, some major challenges still have not been solved in engineered T cells to treat solid tumors and have remain significant barriers to its broader clinical application, especially in terms of specificity, persistence, safety, and immunosuppressive microenvironment. Thus, there is a need for improved CAR-based therapy that is reliable, safe, and effective that can be extended toward the treatment of a broader range of tumors, including solid tumor.

### SUMMARY OF THE INVENTION

One aspect of the invention provides a chimeric antigen receptor (CAR) comprising: (1) an antigen-binding domain specific for the extra domain B (EDB) of fibronectin; (2) a transmembrane (TM) domain of a membrane protein selected from CD3, CD4, CD8, CD28, OX40, or CD137; and, (3) an intracellular ITAM (Immunoreceptor Tyrosine-based Activation Motif) domain of CD3ζ, with or without a costimulatory domain; wherein the CAR, when expressed on the surface of a T cell, is capable of activating the T cell (a) upon binding to a soluble EDB, (b) upon binding to a membrane-bound EDB, and/or (c) upon binding to EDB in extracellular matrices (e.g., those that are part of fibronectin mesh functioning as scaffold for cell attachment).

In certain embodiments, the antigen-binding domain is an scFv, a single chain antibody, a nanobody (e.g., a derivative of VHH (camelid Ig)), a domain antibody (dAb, a derivative of VH or VL domain), a Bispecific T cell Engager (BiTE, a bispecific diabody); a Dual Affinity ReTargeting (DART, a bispecific diabody); an anticalin (a derivative of Lipocalins); an adnectin (10th FN3 (Fibronectin)); a Designed Ankyrin Repeat Proteins (DARPins); or an avimer.

In certain embodiments, the antigen-binding domain is a human scFv or a humanized scFv.

In certain embodiments, the CAR further comprises a hinge/spacer domain between the antigen-binding domain and the TM domain.

In certain embodiments, the hinge/spacer domain and the TM domain originate from the same protein.

In certain embodiments, the same protein is CD8α, and wherein the hinge/spacer domain is the extracellular domain of CD8α.

In certain embodiments, (3) comprises the costimulatory domain.

In certain embodiments, the costimulatory domain is from CD28.

In certain embodiments, (3) comprises two costimulatory domains.

In certain embodiments, the two costimulatory domains comprises a costimulatory domain from CD28, and/or a costimulatory domain from CD27, 4-1BB, or OX-40.

In certain embodiments, the CAR comprises the scFv of residues 21-236 of SEQ ID NO: 1, a CD8α extracellular and transmembrane domain, a 4-1BB intracellular domain, and a CD3ζ intracellular domain.

In certain embodiments, the CAR further comprises an N-terminal signal peptide sequence (such as the hIL-2 signal peptide sequence, or residues 1-20 of SEQ ID NO: 1).

In certain embodiments, the CAR comprises a polypeptide of SEQ ID NO: 1.

Another aspect of the invention provides polynucleotide encoding the CAR of the invention. For example, the polynucleotide may be SEQ ID NO: 2.

In certain embodiments, the polynucleotide is codon-optimized for expression in a human cell.

Another aspect of the invention provides a vector comprising the polynucleotide of the invention.

In certain embodiments, the vector is a viral vector capable of infecting and/or expressing said CAR in T cells, macrophages, and/or NK cells, such as primary human T cells, macrophages, or NK cells.

In certain embodiments, the vector is a viral vector capable of infecting and/or expressing said CAR in peripheral monocytes, monocyte derived dendritic cells, hematopoietic stem cells, and/or induced PSC (pluripotent stem cell).

In certain embodiments, the vector is a lentiviral vector.

In certain embodiments, the lentiviral vector is a self-inactivating lentiviral vector.

Another aspect of the invention provides a cell expressing the CAR of the invention, comprising the polynucleotide of the invention, or the vector of the invention.

In certain embodiments, the cell is an immune cell.

In certain embodiments, the cell is a T cell. In certain embodiments, the cell is an NK cell. In certain embodiments, the cell is a monocyte or a macrophage.

In certain embodiments, the cell is a primary cell isolated from a patient.

In certain embodiments, the cell is from an established cell line, such as an allogeneic cell line with respect to a patient to whom the cell is to be administered.

In certain embodiments, the cell expresses a cytokine.

In certain embodiments, the cytokine comprises IL-2, IL-7, IL-12, IL-15, or IL-21.

In certain embodiments, expression of the cytokine is under the control of a promoter that is activated by activation of the immune cell.

In certain embodiments, the cell further comprises a safety switch for down-regulating the activity of the immune cell.

In certain embodiments, the safety switch comprises a coding sequence for an iCaspase9 (inducible caspase-9) monomer that can be activated by dimerization with, e.g., FKBP, to trigger apoptosis of the immune cell.

Another aspect of the invention provides a method of inhibiting angiogenesis in a subject having a disease or condition treatable by angiogenesis inhibition, the method comprising administering to the subject a therapeutically effective amount of an immune cell expressing a chimeric antigen receptor (CAR) comprising: (1) an antigen-binding domain specific for the extra domain B (EDB) of fibronectin; (2) a transmembrane (TM) domain of a membrane protein selected from CD3, CD4, CD8, CD28, OX40 or CD137; and, (3) an intracellular ITAM (Immunoreceptor Tyrosine-based Activation Motif) domain of CD3ζ, with or without a costimulatory domain.

In certain embodiments, the CAR is any one of the CAR described herein.

In certain embodiments, the disease or condition is a solid tumor or a chronic inflammatory condition.

In certain embodiments, cancer cells from the solid tumor do not express EDB on cell surface.

In certain embodiments, the disease or condition is a solid tumor, and wherein the method further comprises administering an immune checkpoint inhibitor such as a PD-1 inhibitor (e.g. pembrolizumab, nivolumab, and cemiplimab), a PD-L1 inhibitor (e.g. atezolizumab, avelumab, and durvalumab), a CTLA-4 targeting agents (e.g. ipilimumab), or an immunomodulating agent (e.g. thalidomide and lenalidomide).

In certain embodiments, the chronic inflammatory condition is psoriasis, rheumatoid arthritis, Crohn's disease, psoriatic arthritis, ulcerative colitis, osteoarthritis, asthma, pulmonary fibrosis, IBD, inflammation-induced lymphangiogenesis, obesity, diabetes, retinal neovascularization (RNV), diabetic retinopathy, choroidal neovascularization (CNV), age-related macular degeneration (AMD), metabolic syndrome-associated disorder, prolonged peritoneal dialysis, juvenile arthritis, or atherosclerosis.

In certain embodiments, the method further comprises administering a second therapeutic agent effective to inhibit angiogenesis.

In certain embodiments, the second therapeutic agent comprises axitinib, bevacizumab, cabozantinib, everolimus, lenalidomide, pazopanib, ramucirumab, regorafenib, sorafenib, sunitinib, thalidomide, vandetanib, and/or ziv-aflibercept.

In certain embodiments, the immune cell is produced by introducing in vitro a vector of the invention into a primary immune cell isolated from the subject, and optionally culturing and/or expanding in vitro the primary immune cells introduced by the vector.

In certain embodiments, the method further comprises administering a reagent that suppresses cytokine release syndrome (CRS), such as an anti-IL-6 monoclonal antibody (e.g., tocilizumab); and/or immunoglobulin therapy.

It should be understood that any one embodiment of the invention described herein, including any one embodiment described only in the examples or claims, can be combined with any one or more other embodiments of the invention, unless expressly disclaimed or otherwise improper.

### BRIEF DESCRIPTION OF THE DRAWINGS

FIG. 1 shows expression of EDB-CAR on lentivirus-transduced human T cells in flow cytometry analysis. M1: mock transduction T cells. T: untransduced T cells.

FIG. 2A shows production of IFN-7 by EDB-CAR T cells in the presence of recombinant EDB protein.

FIG. 2B shows lysis of U87-MG cells after 2-24 hours co-culturing with EDB-CAR T cells at effector to target (E:T) ratio of 5:1. Cell lysis was determined using an LDH assay. N=3, and each data point reflects the mean SEM of triplicates (*, P<0.05; **, P<0.01; ***, P<0.001; two-tailed Student's t-test).

FIGS. 3A-3B show levels of expression of EDB in multiple cell lines detected at protein level by Western blotting (FIG. 3A) and at mRNA level by qPCR (FIG. 3B).

FIGS. 4A-4B show cytotoxicity of EDB-CAR T cells to human or murine cancer cells and HUVEC cells after co-culturing for 24 hours at various E:T ratios. Lysis of target cells was determined using an LDH assay. N=3; two-tailed Student's t-test.

FIG. 5 shows in vitro IFN-7 production by EDB-CAR T cells in the presence of tumor cells. EDB CAR-T cells were incubated for 24 hours with cancer cells at various E:T ratios for 24 hours. Supernatants were assayed for IFN-7. N=3; two-tailed Student's t-test.

FIG. 6 shows TNF-α production by EDB-CAR T cells in the presence of tumor cells. EDB-CAR T cells were incubated for 24 hours with the cancer cells at various E:T ratios for 24 hours. Supernatants were assayed for TNF-α. N=3; two-tailed Student's t-test.

FIG. 7 shows expression of EDB-CAR on NK-92 after transduction by flow cytometry analysis.

FIG. 8A shows cytotoxicity of EDB-CAR NK-92 cells, and FIG. 8B shows release of IFN-7 to the supernatant by EDB-CAR NK-92 cells after co-culturing with U87-MG cells at various E:T ratios for 24 hours. N=3; two-tailed Student's t-test.

FIG. 9 shows the results of histopathological analysis of murine organ tissues by hematoxylin and eosin staining, demonstrating the lack of pathological changes/toxicity in normal mice injected with very high doses of CAR-T cells specific for EDB. The images were taken through Leica Aperio VERSA 8 slice scanner under magnification×20. Each scale bar represents 100 m.

FIGS. 10A-10B show purity and EDB-CAR-expression of CD14⁺ monocytes. Specifically, FIG. 10A shows the purity of the CD14⁺ monocytes/macrophages (as evidenced by flow cytometry) isolated from PBMCs using CD14 MicroBeads. FIG. 14B shows expression of EDB-CAR in lentiviral-transduced human monocytes, as analyzed by flow cytometry. M1 stands for mock transduction negative control. Transduction efficiency was also shown.

FIGS. 11A-11H, 11J show characterization of EDB-Targeted CAR monocytes. Specifically, EDB-CAR-monocytes/macrophages were incubated with various EDB-expressing cell lines at the various effector:target (E:T) ratios (FIGS. 11A-11F) or 5 μg/mL EDB protein (FIGS. 11G, 11H, 11J) for 24 hours. Culture supernatants were harvested at 24 hrs after cocultured cells were assayed for TNF-α (FIGS. 11C, 11F, and 11J), IL-12 (FIGS. 11B, 11E, and 11H), and IFN-7 (FIGS. 11A, 11D, and 11G) expression. Data are representative of three independent experiments. Each data point reflects the mean SEM of triplicates (*: P<0.05; **: P<0.01; ***: P<0.001; two-tailed Student t test).

FIG. 12 shows expression of EDB-CAR (SEQ. ID 1-17) in primary human T cells in flow cytometry analysis. M1: mock transduction T cells. T: untransduced T cells.

FIG. 13 shows that repeated stimulation of EDB-CAR (SEQ. ID 1, 3-7) transduced primary human T cells can activate proliferation of the transduced cells. The proliferation was determined by cell counting. M1: mock transduction T cells. T: untransduced T cells.

FIG. 14 shows that EDB-CAR (SEQ. ID NO 1, 3-7) transduced primary human T cells were cytotoxic to the U87MG cancer cells. M1: mock transduction T cells. T: untransduced T cells.

FIG. 15 shows that repeated stimulation of EDB-CAR (SEQ. ID NO 1, 3, 18) transduced primary human T cells can activate proliferation of the transduced cells. Note the proliferation was determined by labeling of the T cells with Celltrace dye followed by flow cytometry analysis. M1: mock transduction T cells. T: untransduced T cells.

FIG. 16 shows that EDB-CAR transduced primary human T cells were cytotoxic to the U87MG cancer cells. EDB BB: SEQ ID 1; EDB137pro: SEQ. ID NO 10; EDB-αCD3: SEQ. ID NO 18; EDB-αCD3/EDB BB: co-transduction using SEQ. ID NO 18 and SEQ. ID NO 1; EDB-αCD3/137pro: co-transduction using SEQ. ID NO 18 and SEQ. ID NO 10. M1: mock transduction T cells. T: untransduced T cells.

FIG. 17 shows that repeated stimulation of EDB-CAR transduced primary human T cells can activate proliferation of the transduced cells. EDB BB: SEQ ID 1; EDB28: SEQ. ID NO 3; CD3z: SEQ. ID NO 12; CD3z/28pro: co-transduction using SEQ. ID NO 12 and SEQ. ID NO 9; CD3z/137pro: co-transduction using SEQ. ID NO 12 and SEQ. ID NO 10; CD3z/CD4CD28: co-transduction using SEQ. ID NO 12 and SEQ. ID NO 8. Note the proliferation was determined by labeling of the T cells with Celltrace dye followed by flow cytometry analysis. M1: mock transduction T cells. T: untransduced T cells.

FIG. 18 shows that EDB-CAR transduced primary human T cells were cytotoxic to the U87MG cancer cells. EDB BB: SEQ ID 1; EDB28: SEQ. ID NO 3; CD3z: SEQ. ID NO 12; CD3z/28pro: co-transduction using SEQ. ID NO 12 and SEQ. ID NO 9; CD3z/137pro: co-transduction using SEQ. ID NO 12 and SEQ. ID NO 10; CD3z/CD4CD28: co-transduction using SEQ. ID NO 12 and SEQ. ID NO 8. M1: mock transduction T cells. T: untransduced T cells.

FIG. 19 shows that repeated stimulation of EDB-CAR transduced primary human T cells can activate proliferation of the transduced cells. EDB BB: SEQ ID 1; EDB28: SEQ. ID NO 3; CD3zFL: SEQ. ID NO 11; CD3eFL: SEQ. ID NO 15. Note the proliferation was determined by labeling of the T cells with Celltrace dye followed by flow cytometry analysis. M1: mock transduction T cells. T: untransduced T cells.

FIG. 20 shows that EDB-CAR transduced primary human T cells were cytotoxic to the U87MG cancer cells. EDB BB: SEQ ID 1; EDB28: SEQ. ID NO 3; CD3zFL: SEQ. ID NO 11; CD3zFL/28pro: co-transduction using SEQ. ID NO 11 and SEQ. ID NO 9; CD3zFL/137pro: co-transduction using SEQ. ID NO 11 and SEQ. ID NO 10; CD3zFL/CD4CD28: co-transduction using SEQ. ID NO 11 and SEQ. ID NO 8; CD3eFL: SEQ. ID NO 15; CD3eFL/28pro: co-transduction using SEQ. ID NO 15 and SEQ. ID NO 9; CD3eFL/137pro: co-transduction using SEQ. ID NO 15 and SEQ. ID NO 10; CD3eFL/CD4CD28: co-transduction using SEQ. ID NO 15 and SEQ. ID NO 8. M1: mock transduction T cells. T: untransduced T cells.

FIG. 21 shows that repeated stimulation of EDB-CAR transduced primary human T cells can activate proliferation of the transduced cells. EDB BB: SEQ ID 1; EDB28: SEQ. ID NO 3; CD3eFLCD28: SEQ. ID NO 16; CD3eFLCD137: SEQ. ID NO 17. Note the proliferation was determined by labeling of the T cells with Celltrace dye followed by flow cytometry analysis as well as by imaging that show aggregate status of proliferative T cells. M1: mock transduction T cells. T: untransduced T cells.

FIG. 22 shows that EDB-CAR transduced primary human T cells were cytotoxic to the U87MG cancer cells. EDB BB: SEQ ID 1; EDB28: SEQ. ID NO 3; CD3eFL/28pro: co-transduction using SEQ. ID NO 15 and SEQ. ID NO 9; CD3eFL/137pro: co-transduction using SEQ. ID NO 15 and SEQ. ID NO 10; CD3eFLCD28: SEQ. ID NO 16; CD3eFLCD137: SEQ. ID NO 17. M1: mock transduction T cells. T: untransduced T cells.

FIG. 23 shows that “Hijack Plus” EDB-CAR based on SEQ. ID NO 16 and 17 were incorporated into TCR complex. Human primary T cells were transduced with “Hijack Plus” EDB-CARs and immune precipitated with biotin-labeled polyclonal goat anti-human-IgG F(ab′)₂ fragment antibodies. CD3ζ subunit of the TCR complex was detected using anti CD3ζ antibodies (FIG. 23 A). Jurkat cells were transduced with “Hijack Plus” EDB-CARs and immune precipitated with biotin-labeled polyclonal goat anti-human-IgG F(ab′)₂ fragment antibodies. CD3ζ subunit of the TCR complex was detected using anti CD3ζ antibodies (FIG. 23 B).

FIG. 24 shows in vivo efficacies of various EDB CAR T cells in treatment of tumors formed by U87MG cells using NCG mice. 2^{nd} generation EDB CAR (FIG. 24A), “trans” EDB CAR (FIG. 24B), bi-specific EDB-αCD3 (FIG. 24C), “hijack” EDB CAR (FIG. 24D), or “hijack plus” EDB CAR (FIG. 24E) showed variable levels of tumor growth inhibition or tumor regression.

### DETAILED DESCRIPTION OF THE INVENTION

### 1. Overview

The invention described herein is partly based on the discovery that certain antibodies or antigen-binding fragments thereof specific for the EDB domain of fibronectin can be used to construct CAR (chimeric antigen receptor) constructs that not only recognizes membrane bound EDB, or EDB in extracellular matrix (these deposits in tumor tissues), but also soluble form of EDB in solution.

The invention described herein is also partly based on the surprising discovery that immune cells bearing the subject EDB-specific CAR (such as CAR T cells) are cytotoxic in vitro against normal human umbilical vein endothelial cells (HUVECs), yet very large amounts of such CAR-bearing immune cells (e.g., T cells) injected in vivo to mice do not elicit expected toxicity. As is known in the art, a prominent barrier to widespread use of CAR T-cell therapy is toxicity, primarily cytokine release syndrome (CRS) and neurologic toxicity. For example, earlier attempts for solid tumor treatment using CAR against Her2 or carboxyanhydrase IX were unsuccessful due to on-target toxicity towards healthy tissues, leading to uncontrolled inflammatory-driven tissue damages or even death. Manifestations of CRS include fevers, hypotension, hypoxia, end organ dysfunction, cytopenias, coagulopathy, and hemophagocytic lymphohistiocytosis. Neurologic toxicities are diverse and include encephalopathy, cognitive defects, dysphasias, seizures, and cerebral edema. Yet such symptoms appear to be absent using the subject CAR constructs.

Thus, the subject CAR constructs have been found to be able to support CAR-based immune therapy using, e.g., CAR T or CAR NK cells, to treat diseases in which angiogenesis is a pathological condition. Such diseases include cancer and inflammatory diseases.

Thus in one aspect, the invention provides a chimeric antigen receptor (CAR) comprising: (1) an antigen-binding domain specific for the extra domain B (EDB) of fibronectin; (2) a transmembrane (TM) domain of a membrane protein, such as one selected from CD3, CD4, CD8, CD28, OX40 or CD137; and, (3) a signaling domain such as an intracellular ITAM (Immunoreceptor Tyrosine-based Activation Motif) domain of CD3ζ, with or without a costimulatory domain; wherein the CAR, when expressed on the surface of a T cell, is capable of activating the T cell (a) upon binding to a soluble EDB, (b) upon binding to a membrane-bound EDB, and/or (c) upon binding to EDB in extracellular matrices (e.g., those that are part of fibronectin mesh functioning as scaffold for cell attachment). A representative CAR of the invention is SEQ ID NO: 1.

Additional representative CARs of the invention are shown in SEQ ID NO: 3, 4, 5, 6, 7, 8, 9, 11, 12, 13, 14, 15, 16, 17.

Another aspect of the invention provides a polynucleotide encoding the CAR of the invention, such as SEQ ID NO: 2.

Another aspect of the invention provides a vector comprising the polynucleotide of the invention, such as a lentiviral vector comprising SEQ ID NO: 2.

Another aspect of the invention provides a cell, such as an immune cell, comprising the CAR of the invention, the polypeptide of the invention, and/or a vector of the invention. The cell may be a T cell, or an NK cell.

Another aspect of the invention provides a method of inhibiting angiogenesis in a subject having a disease or condition treatable by angiogenesis inhibition, the method comprising administering to the subject a therapeutically effective amount of an immune cell expressing a chimeric antigen receptor (CAR) comprising: (1) an antigen-binding domain specific for the extra domain B (EDB) of fibronectin; (2) a transmembrane (TM) domain of a membrane protein selected from CD3, CD4, CD8, CD28, OX40 or CD137; and, (3) an intracellular ITAM (Immunoreceptor Tyrosine-based Activation Motif) domain of CD3ζ, with or without a costimulatory domain.

The disease or condition may be a solid tumor or a chronic inflammatory condition.

With the general aspects of the invention described herein, the following section provide further details regarding the different aspects of the invention.

### 2. EDB of Fibronectin (FN)

Fibronectin is a high-molecular weight glycoprotein of the extracellular matrix (ECM) that binds to membrane-spanning receptor proteins integrins and ECM components such as collagen, fibrin, and heparan sulfate proteoglycans. Fibronectin exists as a protein dimer, consisting of two nearly identical monomers linked by a pair of disulfide bonds. Fibronectin is encoded by a single gene, but alternative splicing of its pre-mRNA leads to the creation of at least 20 different isoforms in humans (see a general discussion of FN function by White and Muro, “Fibronectin splice variants: understanding their multiple roles in health and disease using engineered mouse models.” IUBMB Life. 63(7):538-546, 2011 (incorporated herein by reference).

The FN monomers are each about 250 kDa in size, and are linked together by disulfide bonds near the C-terminus. FNs are made of repeating units of three different types of homologies: type I, II, and III, having about 40, 60, and 90 amino acids, respectively. Many of these independently folded domains are also present in different ECM proteins. Among them, the type III modules are the most abundant modules in the FN molecule, and are also found in many different proteins across a wide range of species, whereas type I modules are found only in vertebrates.

In human, FN protein diversity is obtained by alternative splicing of two type III exons, known as Extra Domains A and B (also called EIIIA and EIIIB), respectively, and of a segment connecting two other type III repeats—type III connecting segment (IIICS). EDA and EDB splicing is similar in all species (either total inclusion or exclusion), whereas that of the IIICS region is species-specific (five variants in humans, three in rodents, and two in chickens).

FN is found either as a soluble dimer in plasma, secreted by hepatocytes directly into circulation (plasma FN, or pFN), or deposited as insoluble fibrils in the ECM of tissues (cellular FN, or cFN). The two FN isoforms differ in the presence of the EDA and EDB domains: (a) pFN lacks the alternatively spliced EDA and EDB sequences and (b) cFN contains variable proportions of these domains.

As used herein, the term “EDB,” “EIIIB,” “EDB domain” or “ED-B-domain” refers to the extra-domain B of (human) fibronectin. In human, EDB is a type III homology domain with about 91 residues. EDB is essentially undetectable in healthy adult tissues, but is highly abundant in the vasculature of many aggressive solid tumors, thus making EDB a suitable target for anti-cancer and/or anti-inflammatory therapy of the invention.

In one embodiment, the antigen recognized by the subject CAR is a splice isoform of fibronectin, such as the ED-B domain of FN.

### 3. Antibody and Antigen-Binding Fragment of EDB

In certain embodiments, the CAR binds to the EDB-domain of fibronectin exhibits a high binding affinity, e.g., with a K_{D} value of nanomolar or subnanomolar. Affinity can be measured using any art-recognized methods, such as by Bilayer Interferometry (BLI), surface plasmon resonance (SPR) or BIACORE, or other methods.

In certain embodiments, the antigen-binding portion of the CAR is based on EDB-specific antibodies or antigen-binding fragments thereof, such as those described in WO99/058570 (all incorporated herein by reference).

In certain embodiments, the EDB-specific antibody or antigen-binding fragments thereof is based on CAA06864.2 (incorporated herein by reference).

In certain embodiments, the EDB-specific antibody or antigen-binding fragments thereof is based on at least one CDR sequence of the L19 antibody.

In certain embodiments, the EDB-specific antibody or antigen-binding fragments thereof is based on huBC1, which is a humanized antibody that targets a cryptic sequence of the human ED-B-containing fibronectin isoform, B-FN, present in the subendothelial extracellular matrix of most aggressive tumors. B-FN is oncofetal and angiogenesis-associated.

In some embodiments, the antigen-binding portion of the CAR comprises an amino acid sequence sharing at least 80%, 81%, 82%, 83%, 84%, 85%, 86%, 87%, 88%, 89%, 90%, 91%, 92%, 93%, 94%, 95%, 96%, 97% 98%, or 99% identity with any of the antigen-binding portion of the CAR amino acid sequences provided herein. In some embodiments, the antigen-binding portion of the CAR may comprise up to 5 (e.g., 4, 3, 2, or 1) amino acid residue variations in one or more of the CDR regions of one of the antibodies exemplified herein, and binds the same epitope of EDB with substantially similar affinity (e.g., having a K_{D} value in the same order or magnitude). In certain embodiments, the amino acid residue variations are conservative amino acid residue substitutions. As used herein, a “conservative amino acid substitution” refers to an amino acid substitution that does not alter the relative charge or size characteristics of the protein in which the amino acid substitution is made.

As used herein, “antibody” or “immunoglobulin (Ig)” generally comprises four polypeptide chains, two heavy chains (HCs) and two light chains (LCs), but also includes equivalent Ig homologues such as camelid (e.g., alpaca) nanobody (which comprises only a heavy chain), single domain antibody (dAb) (which can be derived either from a heavy or a light chain), and also includes full length or functional mutants, variants, or derivatives thereof (including, but not limited to, murine, chimeric, humanized and fully human antibodies, which retain the essential epitope binding features of an Ig molecule, and including dual specific, bispecific, multispecific, and dual variable domain immunoglobulins. Antibody or immunoglobulin can be of any class, e.g., IgG, IgE, IgM, IgD, IgA, and IgY (a type of immunoglobulin which is the major antibody in bird, reptile, and lungfish blood, as well as being in high concentrations in chicken egg yolk), or subclass, e.g., IgG1, IgG2, IgG3, IgG4, IgA1, and IgA2 and allotype.

“Humanized” antibody or antigen-binding fragment thereof results from replacing one or more amino acid residues in the amino acid sequence of the naturally occurring non-human antibody or fragment thereof, such as VHH sequence (and, in particular, in the framework sequences) by one or more of the amino acid residues that occur at the corresponding position(s) in a VH domain from a conventional four-chain antibody from a human being. Methods for humanization are well known. Humanized antibody or antigen-binding fragment thereof may have several advantages, such as a reduced immunogenicity, compared to a corresponding naturally occurring non-human antibody or domain thereof.

“Humanization” can be performed by providing a nucleotide sequence that encodes a naturally occurring antibody, and then changing one or more codons in the nucleotide sequence in such a way that the new nucleotide sequence encodes a “humanized” version thereof. This nucleic acid can then be expressed to provide the humanized antibody or fragment. Alternatively, based on the amino acid sequence of a naturally occurring non-human sequence, humanized version can be designed and then synthesized de novo using techniques for peptide synthesis. The skilled artisan may also combine one or more parts of one or more naturally occurring sequences (such as one or more FR sequences or CDR sequences), and/or one or more synthetic or semi-synthetic sequences, in a suitable manner, so as to provide a nucleotide sequence or nucleic acid encoding the humanized antibody or fragment thereof. Optionally, the humanized sequence is also codon-optimized for expression in an immune cell of the host, such as human T cell, NK cell, monocyte or macrophage.

An “antibody derivative or antigen-binding fragment,” as used herein, includes a molecule comprising at least one polypeptide chain derived from an antibody that is not full length, including, but not limited to (i) a Fab fragment, which is a monovalent fragment consisting of the variable light (VL), variable heavy (VH), constant light (CL) and constant heavy 1 (CH1) domains; (ii) a F(ab′)₂ fragment, which is a bivalent fragment comprising two Fab fragments linked by a disulfide bridge at the hinge region; (iii) a heavy chain portion of a Fab (Fd) fragment, which consists of the VH and CH1 domains; (iv) a variable fragment (Fv) fragment, which consists of the VL and VH domains of a single arm of an antibody, (v) a domain antibody (dAb) fragment, which comprises a single variable domain; (vi) an isolated complementarity determining region (CDR); (vii) a single chain Fv Fragment (scFv); (viii) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites; and (ix) a linear antibody, which comprises a pair of tandem Fv segments (VH-CH1-VH-CH1) which, together with complementarity light chain polypeptides, form a pair of antigen binding regions; and (x) other non-full length portions of immunoglobulin heavy and/or light chains, or mutants, variants, or derivatives thereof, alone or in any combination.

In certain embodiments, the antigen-binding domain is an scFv, a single chain antibody, a nanobody (e.g., a derivative of VHH (camelid Ig)), a domain antibody (dAb, a derivative of VH or VL domain), a Bispecific T cell Engager (BiTE, a bispecific diabody); a Dual Affinity ReTargeting (DART, a bispecific diabody); an anticalin (a derivative of Lipocalins); an adnectin (10th FN3 (Fibronectin)); a Designed Ankyrin Repeat Proteins (DARPins); or an avimer.

In certain embodiments, the antigen-binding domain is a human scFv or a humanized scFv.

In any case, said derivative or fragment retains or substantially retains target binding properties (e.g., K_{D} that is less than 5%, 10%, 20%, 30%, 40%, 50%, 80%, 2-fold, 3-fold, 5-fold, 7-fold, 8-fold, or 10-fold higher than that of the full-length antibody) of the full-length antibody.

In certain embodiments, the antigen-binding fragment of the invention also includes “antibody-based binding protein,” which as used herein, refers to any protein that contains at least one antibody-derived VH (heavy chain variable region), VL (light chain variable region), or CH (heavy chain constant region) immunoglobulin domain in the context of other non-immunoglobulin, or non-antibody derived components. Such antibody-based proteins include, but are not limited to (i) Fc-fusion proteins of binding proteins, including receptors or receptor components with all or parts of the immunoglobulin CH domains, (ii) binding proteins, in which VH and or VL domains are coupled to alternative molecular scaffolds, or (iii) molecules, in which immunoglobulin VH, and/or VL, and/or CH domains are combined and/or assembled in a fashion not normally found in naturally occurring antibodies or antibody fragments.

In certain embodiments, the antigen-binding fragment of the invention also includes “modified antibody format,” which as used herein, encompasses antibody-drug-conjugates, Polyalkylene oxide-modified scFv, Monobodies, Diabodies, Camelid (e.g., alpaca) Antibodies, Domain Antibodies, bi- or tri-specific antibodies, IgA, or two IgG structures joined by a J chain and a secretory component, shark antibodies, new world primate framework+non-new world primate CDR, IgG4 antibodies with hinge region removed, IgG with two additional binding sites engineered into the CH3 domains, antibodies with altered Fc region to enhance affinity for Fe gamma receptors, dimerized constructs comprising CH3+VL+VH, and the like.

In certain embodiments, the antigen-binding fragment of the invention also includes “antibody mimetic,” which as used herein, refers to proteins not belonging to the immunoglobulin family, and even non-proteins such as aptamers, or synthetic polymers. Some types have an antibody-like beta-sheet structure. Potential advantages of “antibody mimetics” or “alternative scaffolds” over antibodies are better solubility, higher tissue penetration, higher stability towards heat and enzymes, and comparatively low production costs. Some antibody mimetics can be provided in large libraries, which offer specific binding candidates against every conceivable target. Just like with antibodies, target specific antibody mimetics can be developed by use of High Throughput Screening (HTS) technologies as well as with established display technologies, just like phage display, bacterial display, yeast or mammalian display. Currently developed antibody mimetics encompass, for example, ankyrin repeat proteins (called DARPins), C-type lectins, A-domain proteins of S. aureus, transferrins, lipocalins, 10th type III domains of fibronectin, Kunitz domain protease inhibitors, ubiquitin derived binders (called affilins), gamma crystallin derived binders, cysteine knots or knottins, thioredoxin A scaffold based binders, SH-3 domains, stradobodies, “A domains” of membrane receptors stabilized by disulfide bonds and Ca²⁺, CTLA4-based compounds, Fyn SH3, and aptamers (peptide molecules that bind to a specific target molecules).

The antigen-binding portion of the CAR specifically recognizing EDB fibronectin, in particular the scFv based on CAA06864.2, can be employed in various antibody formats as described herein. For example, other than scFv, antibody formats based on Fab, (Fab′)₂, diabody, minibody, or nanobody format may be used, based on the CDR sequences of CAA06864.2. In certain embodiments, the antigen-binding fragment thereof is scFv format. In one further embodiment, the heavy and the light chain are connected by a peptide linker.

In certain embodiments, the CAR comprises the sequences according to SEQ ID NO: 1, 3, 4, 5, 6, 7, 8, 9, 10, 11, 12, 13, 14, 15, 16, or 17.

### 4. CAR Specific for EDB

One aspect of the invention provides chimeric antigen receptor (CAR) with an antigen-binding portion specific for the EDB of fibronectin, wherein the CAR, when expressed on the surface of a T cell, is capable of activating the T cell (a) upon binding to a soluble EDB, (b) upon binding to a membrane-bound EDB, and/or (c) upon binding to EDB in extracellular matrices (e.g., those that are part of fibronectin mesh functioning as scaffold for cell attachment).

In certain embodiments, the chimeric antigen receptor comprises an extracellular antigen binding domain, a transmembrane (TM) region, one or more co-stimulatory domain, and an intracellular signal transduction domain. In certain embodiments, the CAR further comprises a hinge/spacer domain between the antigen-binding domain and the TM domain. The hinge and TM domains may originate from the same protein, or from different proteins.

For example, in certain embodiments, the CAR comprises (1) an antigen-binding domain specific for the extra domain B (EDB) of fibronectin (see above); (2) a transmembrane (TM) domain of a membrane protein, such as that from CD3, CD4, CD8, CD28, OX40 or CD137; and, (3) an intracellular ITAM (Immunoreceptor Tyrosine-based Activation Motif) domain of CD3ζ, with or without a costimulatory domain.

In certain embodiments, the extracellular antigen binding region may be an sc-Fv, Fab, scFab or scIgG fragment thereof.

In certain embodiments, the transmembrane region comprises the transmembrane region of CD3ζ, CD4, CD8, CD28, OX40 or CD137.

In some embodiments, the transmembrane region comprises the transmembrane region of a CD28 transmembrane domain.

In some embodiments, the transmembrane region comprises the transmembrane region of a CD8 transmembrane domain, such as CD8α transmembrane domain (e.g., the CD8α hinge region included in SEQ ID NO: 1).

In some embodiments, the CAR further comprises a hinge region between the extracellular antigen binding domain and the transmembrane domain. In certain embodiments, the hinge region is from a CD8 hinge region, such as the CD8α hinge region included in SEQ ID NO: 1.

In certain embodiments, the hinge region and the TM region can be from the same protein, e.g., both from the CD8 protein.

In certain embodiments, the hinge region and the TM region can be from different proteins, e.g., the hinge region may be from the CD8α protein, while the TM region can be from the TM region of CD3 or CD28, etc.

In certain embodiments, the hinge region can be from CD3γ, CD3δ, CD3ε, CD3ζ, CD137 or CD28 protein, while the TM region can be from the TM region of CD3γ, CD3δ, CD3ε, CD3ζ, CD137 or CD28, etc.

In certain embodiments, the hinge and/or transmembrane region of the chimeric receptor allow incorporation of the chimeric proteins into the TCR complex.

In certain embodiments, the chimeric receptors incorporated into the TCR complex can carry additional co-stimulatory signals.

In certain embodiments, the primary T cell activation signals such as derived from CD3γ, CD3δ, CD3ε, and CD3ζ can be found on one polypeptide, while the co-stimulatory signals such as derived from CD28, CD137, and OX40 can be found on another polypeptide.

In certain embodiments, the primary T cell activation signals can be mediated by a bi-specific polypeptide that binds to a tumor antigen and T cell receptor, with or without co-stimulation of the T cells.

In certain embodiments, the primary T cell activation signals can be mediated by a bi-specific polypeptide that binds to a tumor antigen and T cell receptor, while this bi-specific polypeptide can be secreted by the activated T cells or added externally.

In certain embodiments, the length of the hinge region in the CAR is substantially the same as that of the hinge region in SEQ ID NO: 1, 3, 4, 5, 6, 7, 8, 9, 10, 11, 12, 13, 14, 15, 16, or 17. For example, the hinge region may be longer or shorter than the hinge region in SEQ ID NO: 1, 3, 4, 5, 6, 7, 8, 9, 10, 11, 12, 13, 14, 15, 16, or 17 by no more than 10, 9, 8, 7, 6, 5, 4, 3, 2, or 1 residue(s).

In certain embodiments, the CAR comprises one or more signal transduction domain(s) capable of activating the immune cell in which the CAR is expressed.

In certain embodiments, the CAR comprises one or more (e.g., two) signal transduction domain(s) capable of stimulating T-cell activation. In certain embodiments, the one or more signal transduction domain(s) can include, without limitation, one or more of TCRζ, FcRγ, FcRβ, FcRε, CD3γ, CD3δ, CD3ε, CD3ζ, signal transduction domain of CD5, CD22, CD79a, CD79b, and CD66d. In some embodiments, the CAR comprises a CD3ζ signal transduction domain, such as the CD3ζ signal transduction domain in SEQ ID NO: 1.

In certain embodiments, the two costimulatory domains comprise a costimulatory domain from CD28, and/or a costimulatory domain from CD27, 4-1BB, or OX-40.

In some embodiments, the CAR further comprises one or more co-stimulatory domain from one or more of: CD2, CD3, CD4, CD5, CD7, CD27, CD28, CD30, CD40, CD83, CD86, CD127, CD134, CD137/4-1BB, 4-1BBL, OX-40, PD-1, LFA-1, Lck, DAP10, LIGHT, NKG2C, B7-H3, CD3ζ, or ICOS. In certain embodiments, the one or more co-stimulatory domain comprises an intracellular signal transduction region from CD3ζ, FcεFRIγ, PKCθ, or ZAP70. In some embodiments, the CAR comprises a CD28 co-stimulatory domain. In certain embodiments, the CAR comprises intracellular domain from 4-1BB (CD137), which acts as the costimulatory signaling domain of the CAR, and serves to enhance antigen activation and increase potency. In certain other embodiments, the CAR comprises the intracellular domain of CD28, which also increases CAR-mediated T cell activation.

In some embodiments, the CAR further comprises one polypeptide encoding the primary stimulation signal such as CD3ζ, CD3ε, CD3γ, CD3δ, and another polypeptide encoding the co-stimulatory signal from one of more of CD2, CD4, CD5, CD7, CD27, CD28, CD30, CD40, CD83, CD86, CD127, CD134, CD137/4-1BB, 4-1BBL, OX-40, PD-1, LFA-1, Lck, DAP10, LIGHT, NKG2C, B7-H3, or ICOS. In certain embodiments, the one or more co-stimulatory domain comprises an intracellular signal transduction region from CD3ζ, FcεFRIγ, PKCθ, or ZAP70. In this conformation the membrane proximities of the primary or co-stimulatory signaling domains resemble that of the native forms.

In certain embodiments, a leader sequence or signal peptide is fused N-terminal to the CAR to promote CAR secretion. In certain embodiments, the leader sequence of the GM-CSF receptor may be used. In certain embodiments, the leader sequence is that of the human IL-2.

In some embodiments, the CAR further comprises a reporter molecule, such as GFP, for display or tracking CAR expression.

In one embodiment, the CAR comprises the scFv based on CAA06864.2, fused to the CD8α extracellular and transmembrane domains, the 4-1BB intracellular domain, and the CD3ζ intracellular domain. In some embodiments, the CAR comprises the amino acid sequence of SEQ ID NO: 1, 3, 4, 5, 6, 7, 8, 9, 10, 11, 12, 13, 14, 15, 16, or 17. In certain embodiments, the entire EDB CAR is expressed with a signal peptide, such as the human interleukin-2 signal peptide, for directing it to the plasma membrane.

In another embodiment, the CAR comprises the scFv based on CAA06864.2, fused to the CD8α extracellular and transmembrane domains, the CD28 intracellular domain, and the CD3ζ intracellular domain. In some embodiments, the CAR comprises the amino acid sequence of SEQ ID NO: 3.

In another embodiment, the CAR comprises the scFv based on CAA06864.2, fused to the CD8α extracellular and transmembrane domains, the 4-1BB intracellular domain, CD4 intracellular domain and the CD3ζ intracellular domain. In some embodiments, the CAR comprises the amino acid sequence of SEQ ID NO: 4.

In another embodiment, the CAR comprises the scFv based on CAA06864.2, fused to the CD8α extracellular and transmembrane domains, the 4-1BB intracellular domain, CD8 intracellular domain and the CD3ζ intracellular domain. In some embodiments, the CAR comprises the amino acid sequence of SEQ ID NO: 5.

In yet another embodiment, the CAR comprises the scFv based on CAA06864.2, fused to the CD8α extracellular and transmembrane domains, CD4 intracellular domain, the 4-1BB intracellular domain and the CD3ζ intracellular domain. In some embodiments, the CAR comprises the amino acid sequence of SEQ ID NO: 6.

In yet another embodiment, the CAR comprises the scFv based on CAA06864.2, fused to the CD8α extracellular and transmembrane domains, CD8 intracellular domain, the 4-1BB intracellular domain and the CD3ζ intracellular domain. In some embodiments, the CAR comprises the amino acid sequence of SEQ ID NO: 7.

In yet another embodiment, the CAR comprises the scFv based on CAA06864.2, fused to the CD8α extracellular and transmembrane domains, CD4 intracellular domain, and the CD28 intracellular domain. In some embodiments, the CAR comprises the amino acid sequence of SEQ ID NO: 8.

In yet another embodiment, the CAR comprises the scFv based on CAA06864.2, fused to the CD28 extracellular and transmembrane domains and the CD28 intracellular domain (CD28 aa138-220). In some embodiments, the CAR comprises the amino acid sequence of SEQ ID NO: 9.

In yet another embodiment, the CAR comprises the scFv based on CAA06864.2, fused to the 4-1BB extracellular and transmembrane domains and the 4-1BB intracellular domain (CD137 aa160-255). In some embodiments, the CAR comprises the amino acid sequence of SEQ ID NO: 10.

In yet another embodiment, the CAR comprises the scFv based on CAA06864.2, fused to the CD8 hinge domain and CD3ζ extracellular and transmembrane domains, and the CD3ζ intracellular domain. In some embodiments, the CAR comprises the amino acid sequence of SEQ ID NO: 10.

In yet another embodiment, the CAR comprises the scFv based on CAA06864.2, fused to the CD8α extracellular and transmembrane domains, CD3ζ intracellular domain. In some embodiments, the CAR comprises the amino acid sequence of SEQ ID NO: 12.

In yet another embodiment, the CAR comprises the scFv based on CAA06864.2, fused to a short linker (GRASG), followed by the CD3ε extracellular and transmembrane domains, and intracellular domain. In some embodiments, the CAR comprises the amino acid sequence of SEQ ID NO: 13.

In yet another embodiment, the CAR comprises the scFv based on CAA06864.2, fused to a 10-amino acid linker (2XG4S), followed by the CD3ε extracellular and transmembrane domains, and intracellular domain. In some embodiments, the CAR comprises the amino acid sequence of SEQ ID NO: 14.

In yet another embodiment, the CAR comprises the scFv based on CAA06864.2, fused to a 15-amino acid linker (3XG4S), followed by the CD3ε extracellular and transmembrane domains, and intracellular domain. In some embodiments, the CAR comprises the amino acid sequence of SEQ ID NO: 15.

In yet another embodiment, the CAR comprises the scFv based on CAA06864.2, fused to a 15-amino acid linker (3XG4S), followed by the CD3ε extracellular and transmembrane domains, and intracellular domain, and CD28 intracellular domain. In some embodiments, the CAR comprises the amino acid sequence of SEQ ID NO: 16.

In yet another embodiment, the CAR comprises the scFv based on CAA06864.2, fused to a 15-amino acid linker (3XG4S), followed by the CD3ε extracellular and transmembrane domains, and intracellular domain, and 4-1BB intracellular domain. In some embodiments, the CAR comprises the amino acid sequence of SEQ ID NO: 17.

In some embodiments, the CAR constitutes of two separate polypeptide chains comprising the amino acid sequence of SEQ ID NO: 11 and the amino acid sequence of SEQ ID NO: 8.

In some embodiments, the CAR constitutes of two separate polypeptide chains comprising the amino acid sequence of SEQ ID NO: 12 and the amino acid sequence of SEQ ID NO: 8.

In some embodiments, the CAR constitutes of two separate polypeptide chains comprising the amino acid sequence of SEQ ID NO: 13 and the amino acid sequence of SEQ ID NO: 8.

In some embodiments, the CAR constitutes of two separate polypeptide chains comprising the amino acid sequence of SEQ ID NO: 14 and the amino acid sequence of SEQ ID NO: 8.

In some embodiments, the CAR constitutes of two separate polypeptide chains comprising the amino acid sequence of SEQ ID NO: 15 and the amino acid sequence of SEQ ID NO: 8.

In some embodiments, the CAR constitutes of two separate polypeptide chains comprising the amino acid sequence of SEQ ID NO: 11 and the amino acid sequence of SEQ ID NO: 9.

In some embodiments, the CAR constitutes of two separate polypeptide chains comprising the amino acid sequence of SEQ ID NO: 12 and the amino acid sequence of SEQ ID NO: 9.

In some embodiments, the CAR constitutes of two separate polypeptide chains comprising the amino acid sequence of SEQ ID NO: 13 and the amino acid sequence of SEQ ID NO: 9.

In some embodiments, the CAR constitutes of two separate polypeptide chains comprising the amino acid sequence of SEQ ID NO: 14 and the amino acid sequence of SEQ ID NO: 9.

In some embodiments, the CAR constitutes of two separate polypeptide chains comprising the amino acid sequence of SEQ ID NO: 15 and the amino acid sequence of SEQ ID NO: 9.

In some embodiments, the CAR constitutes of two separate polypeptide chains comprising the amino acid sequence of SEQ ID NO: 11 and the amino acid sequence of SEQ ID NO: 10.

In some embodiments, the CAR constitutes of two separate polypeptide chains comprising the amino acid sequence of SEQ ID NO: 12 and the amino acid sequence of SEQ ID NO: 10.

In some embodiments, the CAR constitutes of two separate polypeptide chains comprising the amino acid sequence of SEQ ID NO: 13 and the amino acid sequence of SEQ ID NO: 10.

In some embodiments, the CAR constitutes of two separate polypeptide chains comprising the amino acid sequence of SEQ TD NO: 14 and the amino acid sequence of SEQ ID NO: 10.

In some embodiments, the CAR constitutes of two separate polypeptide chains comprising the amino acid sequence of SEQ TD NO: 15 and the amino acid sequence of SEQ ID NO: 10.

In some embodiments, a bispecific molecule comprises the scFv based on CAA06864.2 and an ScFv based on an anti CD3ε antibody. The amino acid sequence of SEQ TD NO: 18 exemplify such a polypeptide that can bind to EDB antigen and T-cell receptor.

The nomenclatures of all chimeric antigen receptors disclosed in the current invention with the sequence ID numbers, short descriptions and names are summarized in Table 1.

TABLE 1
Sequence ID NO of the chimeric antigen
receptors and nomenclatures

| SEQ ID NO | Brief Description | Name |
|---|---|---|
| SEQ. 1 | EDB137ic | EDB BB |
| SEQ. 2 | DNA sequence for EDB137ic | |
| SEQ. 3 | EDB28ic | EDB28 |
| SEQ. 4 | EDB 41BB-CD4ic-CD3z | BBCD4CD3z |
| SEQ. 5 | EDB 41BB-CD8ic-CD3z | BBCD8CD3z |
| SEQ. 6 | EDB CD4ic-BB-CD3z | CD4BBCD3z |
| SEQ. 7 | EDB CD8ic-BB-CD3z | CD8BBCD3z |
| SEQ. 8 | EDB CD4ic-CD28ic | CD4CD28 |
| SEQ. 9 | EDB CD28 aa138-220 | 28pro |
| SEQ. 10 | EDB CD137 aa160-255 | 137pro |
| SEQ. 11 | EDB CD3zFL | CD3zFL |
| SEQ. 12 | EDB CD3zic | CD3z |
| SEQ. 13 | EDB 5aaCD3eFL | 5aa CD3eFL |
| SEQ. 14 | EDB 10aalinker CD3eFL | 10aa CD3eFL |
| SEQ. 15 | EDB 15aalinker CD3eFL | CD3eFL |
| SEQ. 16 | EDB 15aalinker CD3eFL CD28ic | CD3eFLCD28 |
| SEQ. 17 | EDB 15aalinker CD3eFL CD137ic | CD3eFLCD137 |

### 5. Polynucleotide

Another aspect of the invention provides a polynucleotide encoding the CAR of the invention described herein. In one embodiment, the polynucleotide comprises SEQ ID NO: 2.

In some embodiments, the nucleic acid is a synthetic nucleic acid. In some embodiments, the nucleic acid is a DNA molecule. In some embodiments, the nucleic acid is an RNA molecule (e.g., an mRNA molecule encoding the CAR). In some embodiments, the mRNA is capped, polyadenylated, substituted with 5-methyl cytidine, substituted with pseudouridine, or a combination thereof.

In some embodiments, the nucleic acid (e.g., DNA) is operably linked to a regulatory element (e.g., a promoter) in order to control the expression of the nucleic acid. In some embodiments, the promoter is a constitutive promoter. In some embodiments, the promoter is an inducible promoter. In some embodiments, the promoter is a cell-specific promoter. In some embodiments, the promoter is an organism-specific promoter.

Suitable promoters are known in the art and include, for example, a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, and a p-actin promoter.

In one aspect, the present disclosure provides nucleic acid sequences that are at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequences described herein, i.e., nucleic acid sequences encoding the CAR described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

In certain embodiments, the nucleic acid molecule encoding the CAR proteins, derivatives or functional fragments thereof are codon-optimized for expression in a host cell or organism. The host cell may include established cell lines (such as T/NK cells) or isolated primary cells. The nucleic acid can be codon optimized for use in any organism of interest, in particular human immune cells. Codon usage tables are readily available, for example, at the “Codon Usage Database” available at www.kazusa.orjp/codon/, and these tables can be adapted in a number of ways. See Nakamura et al., Nucl. Acids Res. 28:292, 2000 (incorporated herein by reference). Computer algorithms for codon optimizing a particular sequence for expression in a particular host cell are also available, such as Gene Forge (Aptagen; Jacobus, Pa.).

An example of a codon optimized sequence, is in this instance a CAR coding sequence optimized for expression in a eukaryote, e.g., humans (i.e. being optimized for expression in humans), or for another eukaryote, animal or mammal as herein discussed). Whilst this is preferred, it will be appreciated that other examples are possible and codon optimization for a host species other than human, or for codon optimization for specific organs is known. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at http://www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a CAR correspond to the most frequently used codon for a particular amino acid.

In some embodiments, the polynucleotide(s) or nucleic acid(s) of the invention are present in a vector (e.g., a viral vector).

The term “vector” as used herein generally refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art.

In certain embodiments, the vector can be a cloning vector, or an expression vector. The vectors can be plasmids, phagemids, Cosmids, etc. The vectors may include one or more regulatory elements that allow for the propagation of the vector in a cell of interest (e.g., a mammalian cell such as a human immune cell like T/NK cell).

In certain embodiments, the vector is a “plasmid,” which refers to a circular double stranded DNA loop into which additional DNA segments can be inserted, such as by standard molecular cloning techniques.

In certain embodiments, the vector is a viral vector, wherein virally-derived DNA or RNA sequences are present in the vector for packaging into a virus (e.g., retroviruses, lentiviruses, replication defective retroviruses, adenoviruses, replication defective adenoviruses, HSV, and adeno-associated viruses (AAV)). Viral vectors also include polynucleotides carried by a virus for transfection into a host cell.

In certain embodiments, the vector is a lentiviral vector. In certain embodiments, the lentiviral vector is a self-inactivating lentiviral vector. See, for example, Zufferey et al., “Self-Inactivating Lentivirus Vector for Safe and Efficient In vivo Gene Delivery.” J Virol. 72(12): 9873-9880, 1998 (incorporated herein by reference).

In certain embodiments, the vector is based on the Sleeping Beauty (SB) transposon, which has been used as a non-viral vector for introducing genes into genomes of vertebrate animals and for gene therapy. Because the SB system is composed solely of DNA, the costs of production and delivery are considerably reduced compared to viral vectors. SB transposons have been used to genetically modify T cell in human clinical trials.

In certain embodiments, the vector is capable of autonomous replication in a host cell into which they are introduced. In certain embodiments, the vector (e.g., non-episomal mammalian vectors) is integrated into the genome of a host cell upon introduction into the host cell, and thereby are replicated along with the host genome. In certain embodiments, the vector, referred to herein as “expression vector,” is capable of directing the expression of genes to which they are operatively-linked. Vectors for and that result in expression in a eukaryotic cell are “eukaryotic expression vectors.”

In certain embodiments, the vector is a recombinant expression vector that comprises a nucleic acid of the invention in a form suitable for expression of the nucleic acid in a host cell. The recombinant expression vector may include one or more regulatory elements, which may be selected on the basis of the host cells to be used for expression, that is operatively-linked to the nucleic acid sequence to be expressed. Here, “operably linked” means that the nucleotide sequence of interest is linked to the regulatory element(s) in a manner that allows for expression of the nucleotide sequence (e.g., in an in vitro transcription/translation system or in a host cell when the vector is introduced into the host cell).

The term “regulatory element” include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes such as T cells, or NK cells). Regulatory elements may also direct expression in a temporal-dependent manner, such as in a cell-cycle dependent or developmental stage-dependent manner, which may or may not also be tissue or cell-type specific.

In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and Hl promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 3-actin promoter, the phosphoglycerol kinase (PGK) promoter, and the EF1a promoter.

Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit b-globin (Proc. Natl. Acad. Sci. USA., Vol. 78(3), p. 1527-31, 1981).

It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein.

In certain embodiments, the vector is a lentiviral or AAV vector, which can be selected for targeting particular types of cells (e.g., with tissue and/or cell type-specific tropism).

The vectors of the invention can be introduced into a target cell, such as a primary T/NK cell, or an “off-the-shelf” allogeneic T/NK cell, using any of many art-recognized methods, such as transfection, lipid vectors, infection, electroporation, microinjection, parenteral injections, aerosol, gene guns, or use of ballistic particles, etc.

In certain embodiments, transfection includes chemical transfection that introduces the vector by, e.g., calcium phosphate, lipid, or protein complexes. Calcium phosphate, DEAE-dextran, liposomes, and lipoplexes (for oral delivery of gene) surfactants and perfluro chemical liquids for aerosol delivery of gene.

In certain embodiments, lipid vectors are generated by a combination of plasmid DNA and a lipid solution that result in the formation of a liposome, which can be fused with the cell membranes of a variety of cell types, thus introducing the vector DNA into the cytoplasm and nucleus, where the encoded gene is expressed. In certain embodiments, folate is linked to DNA or DNA-lipid complexes to more efficiently introduce vectors into cells expressing high levels of folate receptor. Other targeting moieties can be similarly used to target the delivery of the vectors to specific cell types targeted by the targeting moieties.

In certain embodiments, the vector DNA is internalized via receptor-mediated endocytosis.

In certain embodiments, the vector is a lentiviral vector, and the target cell infection spectrum of the vector is expanded by replacing the genes for surface glycoproteins with genes from another viral genome in the packaging cell lines packaging cell lines (PCL) of the vector.

### 6. Immune Cells

The CAR of the invention can be introduced into various kinds of immune cells for CAR-mediated therapy. The immune cells into which the CAR of the invention can be introduced include T cells, NK cells, monocytes (including peripheral monocytes), monocyte derived dendritic cells, macrophages, hematopoietic stem cells, and/or induced pluripotent stem cell (PSC), etc.

Thus in one aspect, the invention also provides a cell comprising any of the CAR of the invention, polynucleotide encoding the CAR protein, or vector of the invention comprising the polynucleotide of the invention.

In certain embodiments, the cell is a eukaryote. In certain embodiments, the cell is a human cell. In certain embodiments, the cell is an immune cell. In certain embodiments, the cell is a T cell, such as CD4⁺ or CD8⁺ T cell. In certain embodiments, the cell is an NK cell.

In certain embodiments, the cell is a monocyte. In certain embodiments, the cell is a macrophage. In certain embodiments, the cell is a primary cell isolated from a patient into which cell a CAR-expressing vector is to be introduced to express the CAR before the cell is reintroduced to the patient. In certain embodiments, the cell is from a healthy donor into which cell a CAR-expressing vector is to be introduced to express the CAR before the cell is reintroduced to a patient different from the healthy donor. Optionally, the HLA-type of the healthy donor matches that of the patient.

In certain embodiments, the T cells and/or NK cells and/or monocytes and/or macrophages of the present invention can be obtained from a number of non-limiting source by various non-limiting method, comprising peripheral blood mononuclear cells (PBMCs), bone marrow, lymph node tissue, cord blood, thymus tissue, ascites, pleural effusion, spleen tissue, and tumors.

In some embodiments, the immune cells are isolated from patients in need of CAR-based therapy, e.g., from patients diagnosed with cancer or inflammatory disease. In this embodiment, the T cells/NK cells/monocytes/macrophages are autologous.

As used herein, “autologous” refers to cell treatment subject, the cell line or cell population derived from the object.

In some embodiments, the immune cells are isolated from healthy donors that are not the patient in need of treatment. In this embodiment, the immune cells are derived from a heterologous host, preferably from a host that is human leukocyte antigen (HLA)-compatible.

In some embodiments, the T-cells comprise CD4⁺ T cells. In some embodiments, the T-cells comprise CD8⁺ T cells.

The subject CAR T cells can be prepared by any means known in the art. For example, expression constructs such as viral-based vectors (e.g., lentiviral vectors) comprising and capable of expressing the CAR polynucleotides of the invention can be used to transduce the isolated immune cells to obtain the subject CAR-T, CAR-NK etc. cells. One of skill in the art can easily construct expression constructs such as viral vectors suitable for protein expression.

In certain embodiments, the cell (e.g., immune cell) further expresses a cytokine, such as IL-2, IL-7, IL-12, IL-15, or IL-21, or combination thereof. In certain embodiments, expression of the one or more cytokine is activated upon binding of the CAR to its target antigen. In certain embodiments, expression of the cytokine is under the control of a promoter that is activated by activation of the immune cell.

In certain embodiments, the cell further comprises a safety switch for down-regulating the activity of the immune cell.

In certain embodiments, the safety switch comprises a coding sequence for an iCaspase9 (inducible caspase-9) monomer that can be activated by dimerization with, e.g., FKBP, to trigger apoptosis of the immune cell.

### 7. Pharmaceutical Composition and Application Thereof

Another aspect of the present invention provides a pharmaceutical composition for the treatment of a disease or condition, such as cancer or inflammatory disease, which comprises a modified T/NK cell/monocyte/macrophage of the invention and a pharmaceutically acceptable carrier. In addition, the invention further claims modified T/NK/monocyte/macrophage of the invention in preparation for use of the medicine for treating the disease.

As used herein, “pharmaceutically acceptable carrier” includes any and all solvents, dispersion media, coatings, antibacterial and antifungal agents, isotonic and absorption delaying agents, and the like physiologically compatible. In certain embodiments, the carrier is suitable for intravenous, intramuscular, subcutaneous, parenteral, spinal or epidermal administration (e.g., by injection or infusion).

In certain embodiments, the invention provides a method of treating a patient having a solid tumor or an inflammatory condition by administering a CAR-based immune cell (e.g., T cell or NK cell) expressing a CAR of the invention. In another embodiment, the invention provides a method of recruiting immune cells to a solid tumor in a patient by administering a CAR-T or CAR-NK cell expressing a CAR. In some instances, the CAR-T/CAR-NK cells can be administered using lymphocyte infusion. Preferably, autologous lymphocyte infusion is used in the treatment. Autologous PBMCs are collected from a patient in need of treatment and T/NK cells are activated and expanded using the methods described herein and known in the art, and then infused back into the patient.

### 8. Method of Use

Another aspect of the invention provides a method for inhibiting angiogenesis in a subject having a disease or condition treatable by angiogenesis inhibition, such as cancer or inflammatory condition, the method comprising administering to the subject a therapeutically effective amount of an immune cell expressing a chimeric antigen receptor (CAR) comprising: (1) an antigen-binding domain specific for the extra domain B (EDB) of fibronectin; (2) a transmembrane (TM) domain of a membrane protein selected from CD3, CD4, CD8, CD28, OX40 or CD137; and, (3) an intracellular ITAM (Immunoreceptor Tyrosine-based Activation Motif) domain of CD3ζ, with or without a costimulatory domain, or a pharmaceutical composition comprising the immune cell.

As used herein, a “therapeutically effective amount” or “therapeutically effective dose” or “effective amount” means administering a sufficient amount of a substance, compound, material or cell to produce a desired therapeutic effect. Therefore, the administered amount is sufficient to prevent, cure, or ameliorate at least one symptom of, or completely or partially blocking the progression/worsening of the disease or condition. The administered amount is also below a threshold toxicity level, above which could/would cause the subject to terminate or discontinue with the therapy.

For example, the immune cells and pharmaceutical composition comprising the immune cells of the present invention, when administered in an effective amount to the subject, may results in reduced/delayed/eliminated one or more disease symptoms, reduced frequency and/or duration of the symptoms of the disease, or prevent or lessen the pain caused by injury or disability due to the disease. For example, for treatment of tumor, the immune cells and pharmaceutical composition comprising the immune cells of the present invention may inhibit cancer cell growth by at least about 10%, at least about 20%, at least about 30%, at least about 40%, at least about 50%, at least about 60%, at least about 70%, at least about 80%, as compared to an untreated control or control population. The ability of the subject immune cells and pharmaceutical composition comprising the immune cells of the present invention to inhibit tumor growth may be evaluated in a suitable animal model system for predicting curative effect for the human tumor. Alternatively or in addition, the ability to inhibit tumor cell growth may be measured in vitro using model system reasonably correlated to the disease or condition.

The amount and the dosage level of the immune cells in the pharmaceutical composition of the invention may be varied depending on specific patient need, the mode of administration, the type and/or degree of cancer in a subject, the desired therapeutic response, the tolerable toxicity to the patient, as well as other factors deemed relevant by an attending physician. That is, the selected dosage level may depend on a variety of pharmacokinetic factors including the particular composition used, the route of administration, the age of the patient, other pharmaceutical composition used in conjunction, duration and time of administration, rate of excretion or elimination, gender, weight, condition, general health condition and medical history, and like factors of the patient, as is generally known in the medical field. One of ordinary skill in the art can empirically determine the effective amount of the invention without necessitating undue experimentation. Combined with the teachings provided herein, by choosing among the various active immune cells and weighing factors such as potency, relative bioavailability, patient body weight, severity of adverse side-effects and preferred mode of administration, an effective prophylactic or therapeutic treatment regimen can be planned which does not cause substantial toxicity in and of itself and yet is entirely effective to treat the particular subject.

Toxicity and efficacy of the protocols of the present invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD50/ED50. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

In certain embodiments, data obtained from the cell culture assays, animal studies and human studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

In certain embodiments, the CAR in the immune cells is any one of the CARs of the invention described herein.

In certain embodiments, the immune cell is an autologous or allogeneic T cell, NK cell, monocyte, or macrophage.

In certain embodiments, the disease or condition is a solid tumor, a chronic inflammatory condition, atherosclerosis, myocardial infarction, fibrosis, or a wound.

Examples of cancer or solid tumor includes: lung cancer, ovarian cancer, colon cancer, colorectal cancer, melanoma, renal cancer, bladder cancer, breast cancer, liver cancer, lymphoma, hematologic malignancies, head and neck cancer, glioma, gastric cancer, nasopharyngeal carcinoma, laryngeal carcinoma, cervical cancer, uterine body cancer and osteosarcoma.

Examples of other cancers can using the method or pharmaceutical composition of the present invention for treating comprising: bone cancer, pancreatic cancer, skin cancer, prostate cancer, skin or intraocular malignant melanoma, uterine cancer, anal region cancer, testicular cancer, uterine cancer, endometrial cancer, vaginal cancer, vulva cancer, Hodgkin's disease, non-Hodgkin's lymphoma, esophageal cancer, small intestine cancer, endocrine system cancer, thyroid cancer, parathyroid cancer, adrenal cancer, soft tissue sarcoma, urethral cancer, penile cancer, chronic or acute leukemia (including acute myeloid leukemia, myeloid leukemia, acute lymphoblastic leukemia, chronic lymphocytic leukemia), childhood solid tumor, lymphocytic lymphoma, bladder cancer, kidney or ureter cancer, renal cancer, cancer of the central nervous system (CNS), primary CNS lymphoma, spinal tumor, brain stem glioma, pituitary adenoma, Kaposi sarcoma, epidermoid cancer, squamous cell cancer, T-cell lymphoma, cancer induced by environment, comprising asbestos-induced cancer, and a combination of said cancer.

In certain embodiments, the cancer is a solid tumor/cancer. In some embodiments, the cancer is lung cancer such as lung squamous cell carcinoma. In some embodiments, the cancer is ovarian cancer. In some embodiments, the cancer is colon cancer.

In certain embodiments, cancer cells from the solid tumor do not express EDB on cell surface.

In certain embodiments, the method further comprises administering an immune checkpoint inhibitor such as a PD-1 inhibitor (e.g. pembrolizumab, nivolumab, and cemiplimab), a PD-L1 inhibitor (e.g. atezolizumab, avelumab, and durvalumab), a CTLA-4 targeting agents (e.g. ipilimumab), or an immunomodulating agent (e.g. thalidomide and lenalidomide).

In some embodiments, the method further comprises administering to the subject radiotherapy and/or chemotherapy and/or surgery and/or other tumor-targeting drug (e.g., targeting monoclonal antibody of other antigen or small molecule compounds).

In certain embodiments, the chemotherapy includes one or more of all-trans retinoic acid, Actinomycin D, Adriamycin, anastrozole, Azacitidine, Azathioprine, Alkeran, Ara-C, Arsenic Trioxide (Trisenox), BiCNU Bleomycin, Busulfan, CCNU, Carboplatin, Capecitabine, Cisplatin, Chlorambucil, Cyclophosphamide, Cytarabine, Cytoxan, DTIC, Daunorubicin, Docetaxel, Doxifluridine, Doxorubicin, 5-flurouracil, Epirubicin, Epothilone, Etoposide, exemestane, Erlotinib, Fludarabine, Fluorouracil, Gemcitabine, Hydroxyurea, Herceptin, Hydrea, Ifosfamide, Irinotecan, Idarubicin, Imatinib, letrozole, Lapatinib, Leustatin, 6-MP, Mithramycin, Mitomycin, Mitoxantrone, Mechlorethamine, megestrol, Mercaptopurine, Methotrexate, Mitoxantrone, Navelbine, Nitrogen Mustard, Oxaliplatin, Paclitaxel, pamidronate disodium, Pemetrexed, Rituxan, 6-TG, Taxol, Topotecan, tamoxifen, taxotere, Teniposide, Tioguanine, toremifene, trimetrexate, trastuzumab, Valrubicin, Vinblastine, Vincristine, Vindesine, Vinorelbine, Velban, VP-16, and/or Xeloda.

In certain embodiments, the chronic inflammatory condition is psoriasis, rheumatoid arthritis, Crohn's disease, psoriatic arthritis, ulcerative colitis, osteoarthritis, asthma, pulmonary fibrosis, IBD, inflammation-induced lymphangiogenesis, obesity, diabetes, retinal neovascularization (RNV), diabetic retinopathy, choroidal neovascularization (CNV), age-related macular degeneration (AMD), metabolic syndrome-associated disorder, prolonged peritoneal dialysis, juvenile arthritis, or atherosclerosis.

In certain embodiments, the method further comprises administering a second therapeutic agent effective to inhibit angiogenesis.

In certain embodiments, the second therapeutic agent comprises axitinib, bevacizumab, cabozantinib, everolimus, lenalidomide, pazopanib, ramucirumab, regorafenib, sorafenib, sunitinib, thalidomide, vandetanib, and/or ziv-aflibercept.

In certain embodiments, the immune cell is produced by introducing in vitro a vector of the invention into a primary immune cell isolated from the subject, and optionally culturing and/or expanding in vitro the primary immune cells introduced by the vector.

In certain embodiments, the method further comprises administering a reagent that suppresses cytokine release syndrome (CRS), such as an anti-IL-6 monoclonal antibody (e.g., tocilizumab); and/or immunoglobulin therapy.

In certain embodiments, the subject is a human, non-human primate, cow, horse, pig, sheep, goat, dog, cat, or rodent. In certain embodiments, the subject is a human subject. In aspects of the invention pertaining to predictive therapy in cancers, the subject is a human either suspected of/at high risk of having the cancer, or having been diagnosed with cancer. Methods for identifying subjects suspected of having cancer may include physical examination, subject's family medical history, subject's medical history, biopsy, or a number of imaging technologies such as ultrasonography, computed tomography, magnetic resonance imaging, magnetic resonance spectroscopy, or positron emission tomography. Diagnostic methods for cancer and the clinical delineation of cancer diagnoses are well known to those of skill in the medical arts.

### 9. Kits

Another aspect of the invention provides a kit, for the method of preparing the immune cells of the present invention, said kit comprising one or more of: the reagents for isolating immune cells from the patient, medium for culturing and expanding the isolated immune cells, reagents including vectors of the invention for infecting the isolated immune cells for expressing the CAR of the invention, reagents for activating the immune cells (e.g., T cells), reagents for detecting/verifying the expression of the CAR induced to express the CAR of the invention, reagents for determining the presence or absence of EDB in a diseased tissue in a subject (such as reagents for immunohistochemistry or immunofluorescence or other imaging modalities such as noninvasive in vivo imaging modalities such as Immuno-PET/CT), etc.

The kit may further comprise instructions to carry out the process of the invention to produce the CAR-bearing immune cells and uses thereof.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

### EXAMPLES

### Example 1: Generation of EDB-CAR T Cells

The chimeric antigen receptor (EDB-CAR, SEQ ID NO: 1) was designed de novo and the coding sequence (SEQ ID NO: 2) was synthesized by Genewiz. The single chain variable domain (scFv) of the EDB-CAR was based on CAA06864.2, which recognizes the EDB antigen. Specifically, the EDB-CAR was constituted as a fusion of gene fragments, in that the EDB specific scFv was fused to CD8α extracellular and transmembrane domain, 4-1BB intracellular domain, and CD3ζ intracellular domain. The entire EDB-CAR receptor was directed to plasma membrane by using a human interleukin-2 signal peptide. The nucleotide sequence encoding the EDB-CAR (SEQ ID NO: 2) was synthesized de novo by Genewiz.

To express the EDB CAR, the fusion gene DNA fragment was cloned into lentiviral vector M1, and the pseudotyped lentivirus was transduced into activated T cells.

T cells were isolated from peripheral blood mononuclear cells (PBMC) using negative magnetic selection method according to protocol as provided by manufacturer (Miltenyi, 130-096-535). T cells were activated with magnetic beads coupled with anti-human CD3 and CD28 antibodies (Thermo Fisher Scientific, 11131D) for 24 hours in complete RPMI (RPMI supplemented with 10% heat-inactivated fetal bovine serum and 100 U/mL Penicillin/streptomycin), 500 U/mL recombinant human TL-2 (SinoBiological, GMP-11848-HNAE), 10 ng/mL TL-7 (SinoiBological, GMP-11840-HNAE), and 10 ng/mL IL-15 (SinoBiological, GMP-11846-HNAE) media and then spin-fected using the lentivirus vector mixed with FuSure (Boston 3T Biotechnologies). Cells were expanded for 12 days and used for in vitro assays.

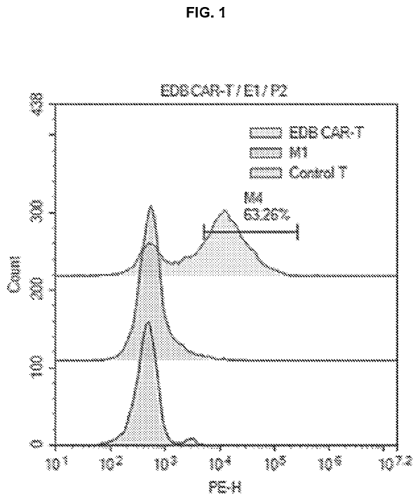

The expression of the EDB-CAR on the surface of the T cells was detected by flow cytometry using an Fab fragment recognizing the human variable chain framework sequences. Specifically, 10⁶ transfected cells were incubated with 8 μg/mL reconstituted biotin-labeled polyclonal goat anti-human-IgG F(ab′)₂ fragment antibodies (Jackson Immunoresearch, Cat #109-066-097) in FACS buffer (PBS plus 0.4% FBS) for 25 min at 4° C. Cells were washed with FACS buffer, and incubated with 5.5 μL Phycoerythrin (R-Phycoerythrin Streptavidin, Jackson Immunoresearch, 016-110-084) in FACS buffer for 20 min on ice in dark. Cells were washed 3 times with ice-cold FACS buffer and analyzed by ACEA Novocyte Flow Cytometer. The Fab fragment only recognized T-cells transduced with the EDB chimeric receptor (FIG. 1).

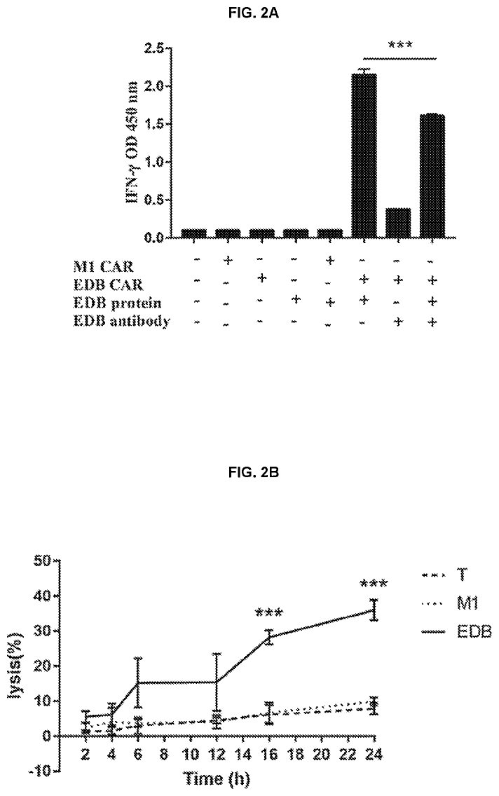

Furthermore, it was discovered that the EDB-CAR transduced T cells recognize soluble EDB antigens and produced IFG-γ, suggesting that the EDB-CAR in this design was activated by soluble antigens. Notably, EDB-specific antibody partially inhibited IFN-γ induction (FIG. 2A). Further, the EDB-CAR T cells were capable of killing cells bearing EDB antigens on surface of U87-MG cells (FIG. 2B).

Thus, the present design of the EDB-CAR is unique in that both soluble and membrane-bound antigens can stimulate the receptors, leading to activation of T cells.

### Example 2: EDB-CAR T Cells are Cytotoxic to EDB-Expressing Cancer Cells

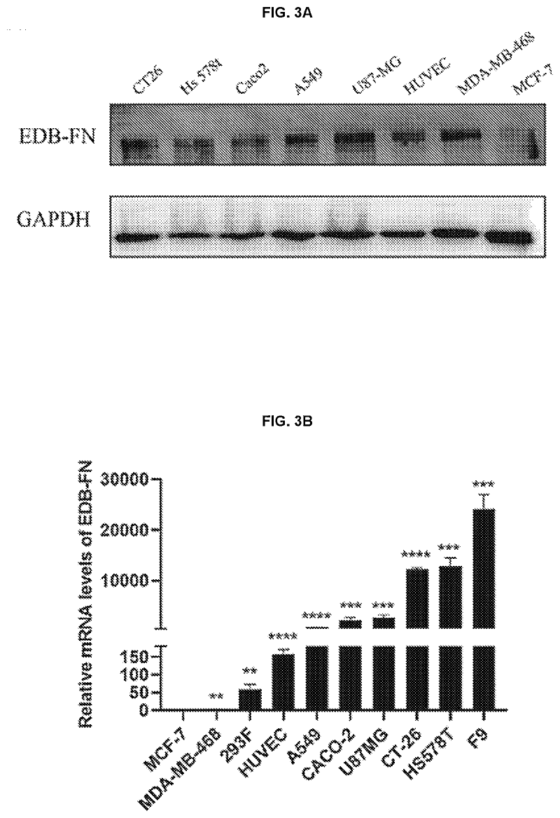

To determine the presence of the EDB domain containing fibronectin in target cells, standard Western blot analysis were performed with anti-EDB monoclonal antibody BC-1 (Abcam, ab154210) followed by secondary antibody detection. EDB was expressed in several representative human cancer cell lines, including Caco-2 for colon adenocarcinoma, MCF-7 breast cancer, HS578T for carcinosarcoma, U87-MG, for human glioblastoma, and MDA-MB-468 for metastatic adenocarcinoma. Murine colorectal cancer cell line CT26 and HUVEC cells also expressed EDB (FIG. 3A).

To substantiate the Western blot analysis, EDB-specific mRNA levels in various cells were confirmed by qPCR using probes specific for the EDB domain. Total RNA from target cells were extracted (19221, YEASEN) and reverse transcribed using total RNA as template (11121ES60, YEASEN). Using the cDNA as template, qPCR reactions were carried out using primers: GADPH F-primer 5′-ACCCAGAAGACTGTGGATGG-3′ and R-primer 5′-TCTAGACGGCAGGTCAGGTC-3′, and EDB F-primer 5′-AAC TCA CTG ACC TAA GCT TT-3′ and R-primer 5′-CGT TTG TGT CAG TGT AG-3′, and SYBR Green dye (11199ES03, YEASEN). The data showed that EDB-specific mRNA was present at varying levels in multiple cancer cell lines and HUVEC, but not detectable in MCF-7 and MDA-MB-468 (FIG. 3B).

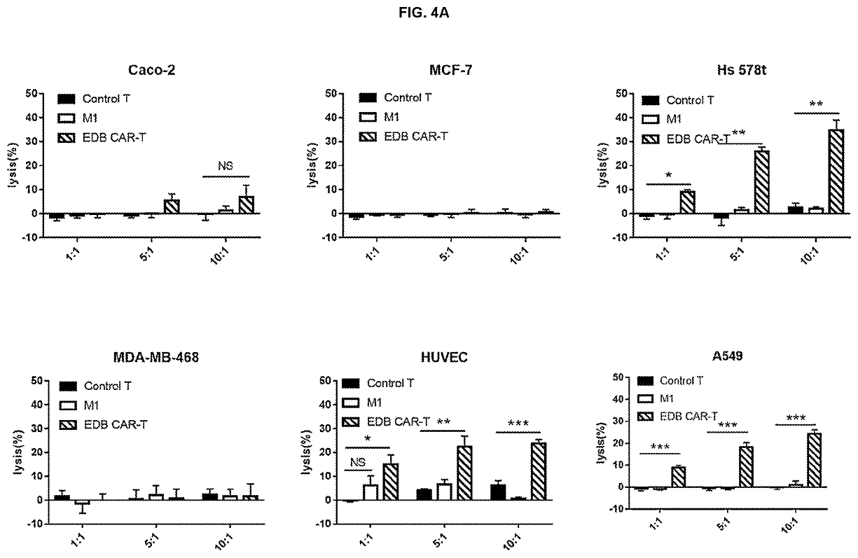

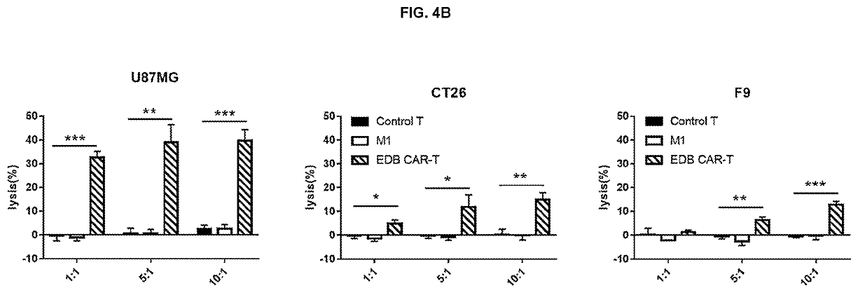

Cytotoxicity of EDB-CAR T cells were tested on a panel of cells that showed variable levels of EDB. Ten thousand target cells were mixed with transduced T cells at effector to target ratios of 1:1, 5:1 and 10:1 in 96-well U-bottom plate. After 24 h culture, target cells lysis was detected by LDH detection kit (Yeasen, 40209ES76). Consistent with the expression analysis (FIGS. 3A-3B), HUVEC, U87-MG, Hs578, A549, F9 were susceptible to EDB-CAR T induced cell lysis, while MCF-7 and MDA-MB468 were not affected (FIGS. 4A-4B). For Caco-2 cell line, the low level of cell killing was probably due to the tendency of the cells forming aggregates in vitro (data not shown).

Interestingly, there were no apparent correlation between EDB expression and the levels of cell lysis. While not wishing to be bound by any particular theory, it is possible that the accessibility or specific conformation of the EDB domain in these cell lines varied, and might affect the susceptibility of the cells to the lysis.

Overall, the data supports cancer treatment using the CAR T cells targeting EDB antigen.

### Example 3: EDB-CAR T Cells are Cytotoxic to HUVEC Endothelial Cells

Angiogenesis is prerequisite for tumor growths and metastasis. Targeting angiogenesis for therapeutic development has resulted in successful demonstrations of therapeutic efficacies for bevacizumab and aflibercept (Keating 2014, Syed 2015).

This example demonstrates that the subject CAR T cells can be used for therapeutic targeting of angiogenesis, based on cytotoxicity of EDB-CAR T cells towards HUVEC cells. HUVEC cells are endothelial cells capable of forming tubular structures. The levels of cytotoxicity of EDB-CAR T cells increased with higher effector to target ratio (FIG. 4A, bottom row, middle).

This suggest that CAR T cells targeting EDB may be an effective angiogenesis inhibitor for treatment of diseases where neovascular generation play a critical role. Due to the involvement of the EDB⁺ fibronectin during the neovascular structure formation, CAR T cells targeting EDB may be used alone, or as combination therapies with current available angiogenesis inhibitors such as bevacizumab or aflibercept.

### Example 4: EDB CAR-T Cells are Activated by EDB-Expressing Cancer Cells

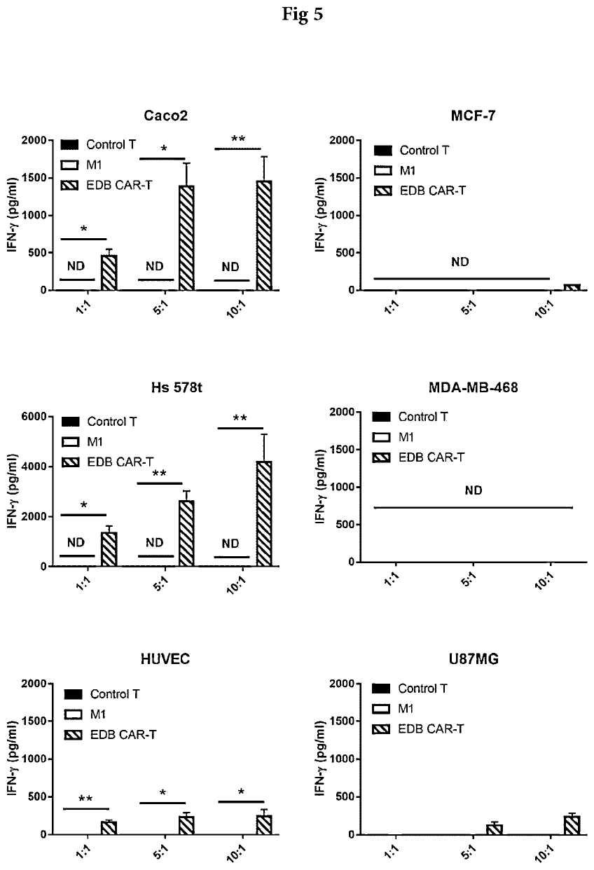

IFN-γ is a hallmark for T cell activation. To assess whether the EDB-CAR T cells based on Sequence ID NO 1 were activated during the cytotoxic reactions, IFN-γ expression were determined by the EDB-CAR T cells by ELISA. Indeed, IFN-γ was found in the culture supernatant in the presence of the target cells (FIG. 5). IFN-γ induction was not observed for MCF-7 and MDA-MB468 cells, consistent with the low or undetectable expression levels of EDB in these cells. These findings strongly suggest that the cell lysis induced by EDB-CAR T cells was due to the binding to the EDB-containing fibronectins.

Interestingly, Caco-2 cells induced high levels of IFN-γ, although the cytotoxicity was low (FIG. 2). This finding is surprising, and is inconsistent with the common observations of cytotoxic activities, and suggests that the IFN-γ induction pathway may be independent of the cytotoxicity pathway for the EDB-CAR T cells.

Indeed, preliminary data suggests that the subject EDB-CAR is likely not proliferative upon cytotoxic reactions, which is quite surprising. On the other hand, this data suggests that it is safer to use the subject EDB-CAR for treatment.

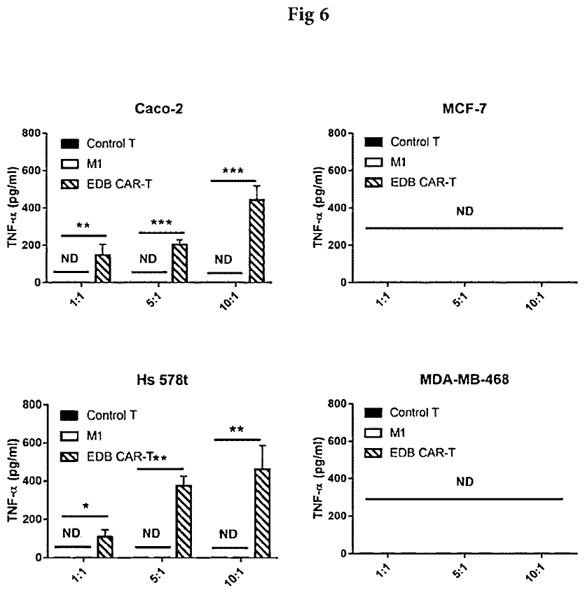

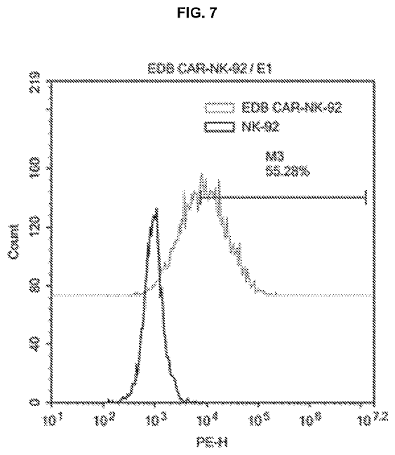

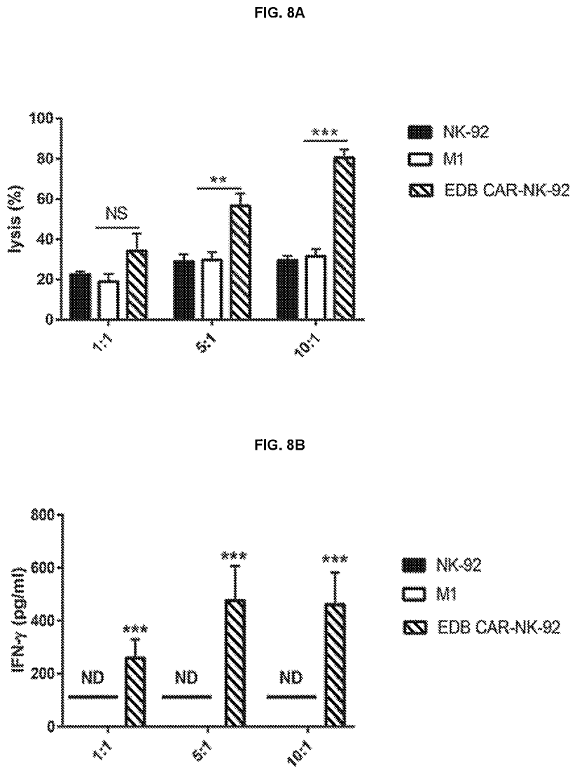

It has also been observed that CAR T cells can produce TNF-α upon cytotoxic killings (Jiang et al 2018). TNF-α induction upon the incubation of the target cells with EDB-CAR T cells was tested. Consistent with IFN-γ expression, incubation with Caco-2 and HS578T cells induced production of TNF-α, which is enhanced at higher effector to target ratios, while no significant amount of TNF-α was produced upon incubation with MCF-7 and MDA-MB-468 cells (FIG. 6).

### Example 5: EDB-CAR Natural Killer Cells are Also Cytotoxic to Cancer Cells

This experiment demonstrates the potential application of natural killer (NK) cell-based treatment options using the subject EDB-CAR based on Sequence ID NO 1.

NK-92 cell line is an immortal cell line derived from a patient and had been used in clinical studies. To demonstrate the applicability of the EDB-CAR for NK cell-based therapies, we transduced the NK-92 cell line with the subject EDB-CAR-expressing lentiviral vector, in a way similar to that used in the generation of the CAR T cells. EDB-CAR expression was analyzed by flow cytometry using the Fab fragment as previously described. Over 55% of the NK-92 cells showed expression of EDB-CAR on the cell surface (FIG. 7).

Cell lysis was induced in U87-MG, a glioblastoma cell line, by co-incubation with EDB-CAR NK-92 cells (FIG. 8A). Consistent with the cytotoxicity assay, IFN-γ was produced by the EDB-CAR NK-92 cells upon activation by target cells.

These results clearly demonstrate the potential utility of the natural killer cells for cancer therapies when transduced with CAR targeting EDB.

### Example 6: EDB-CAR T Cells Injected In Vivo

Since the CAR-T cells have been shown to be cytotoxic to HUVEC cells in vitro, there is a concern that such EDB-specific CAR-T cells may carry unacceptable toxicity in vivo against normal blood vessels in a patient. This experiment demonstrated that the subject CAR-T cells are safe to use in vivo, despite the in vitro cytotoxicity against HUVEC cells.

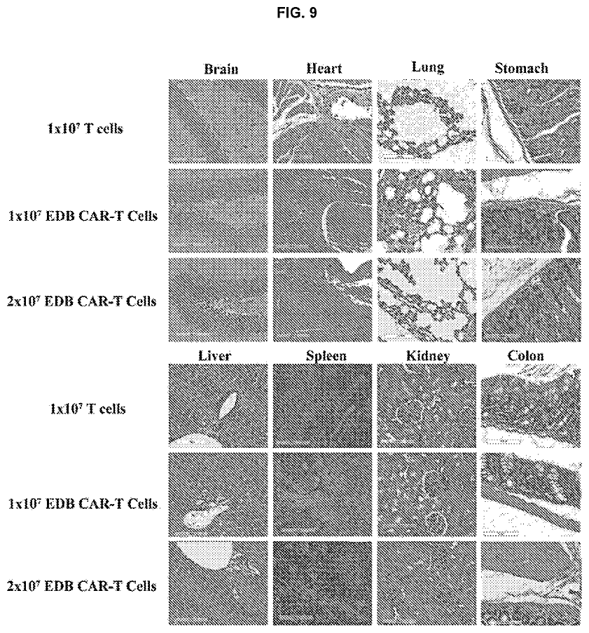

In this experiment, mice were injected either with 1×10⁷ T cells, 1×10⁷ EDB CAR-T cells, or 2×10⁷ EDB CAR-T cells. Mice were sacrificed on days 21 following T-cell infusion. Different tissues were harvested, formalin-fixed, paraffin-embedded, and stained with H&E. Representative photomicrographs are shown in FIG. 9. The images were taken through Leica Aperio VERSA 8 slice scanner under magnification×20. Each scale bar represents 100 m.

The results showed that there was no obvious pathological changes in all tissues examined, and there were no significant differences among all groups. See FIG. 9.

In this experiment, 20 million EDB-specific CAR-T cells were at the very high end of injectable CAR-T cells for a 20 gram or so mouse, which amount of CAR-T is equivalent to about 60 billion cells in a typical 60 kg human, a very high dose that probably cannot be reached in practice.

### Example 7: EDB-CAR T Cells Injected In Vivo

Although CAR-mediated immune therapy has gained wider therapeutic usage in recent years, the understanding of the precise mechanisms through which such immune therapy kills cancer cells remains incomplete. It has recently been reported that long-term killing capability, but not secretion of conventional cytokines or standard 4-hr cytotoxicity, correlates positively with the quality of the CAR-mediated immunological synapse (IS) in two different CAR T cells that share identical antigen specificity, and thus the quality of the IS has been proposed as being predictive of the effectiveness of CAR-modified immune cells. Xenograft model data also confirmed that the quality of the IS in vitro correlates positively with performance of CAR-modified immune cells in vivo. See Xiong et al., Molecular Therapy 26(4):963-975, 2018.

This example, however, demonstrates that EDB-CAR-transduced macrophages produce TNF alpha, suggesting that the EDB-CAR-mediated killing of cancer cells may be partially through the secretion of certain anti-tumor cytokines, and may be less or not dependent on a mechanism that involves immunological synapse, as is typical in CAR-T-mediated killing of cancer cells.

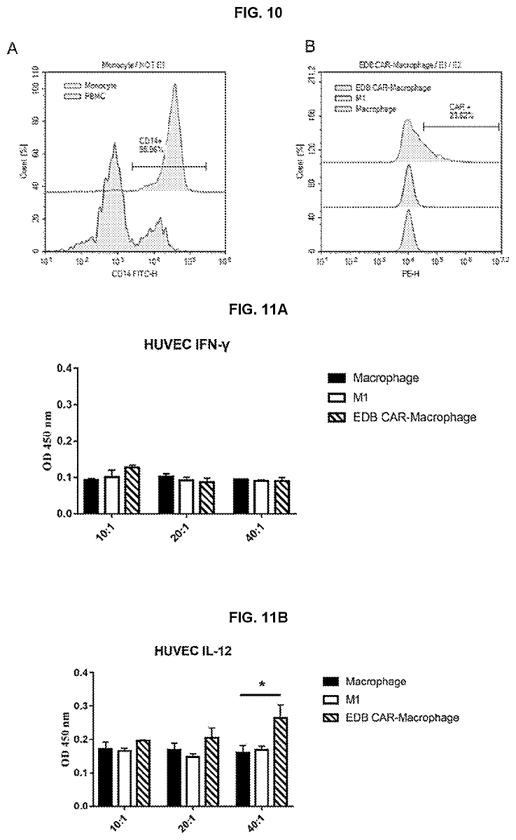

In this experiment, monocytes were isolated from PBMC using positive magnetic selection method according to protocol provided by the manufacturer (130-050-201, Miltenyi). Selected CD14⁺ monocytes were seeded in non-treated cell culture flasks in RPMI with 10% FBS and 10 ng/mL recombinant human GM-CSF (300-03-20, PeproTech) for 8 days, before spin-fecting using the SEQ ID NO: 1 EDB-CAR-expressing lentiviral vector mixed with FuSure (Boston 3T Biotechnologies) at day 6. Monocytes/macrophages were harvested at day 7 and tested for expression of EDB-CAR (SEQ ID NO: 1). See FIGS. 10A-10B.

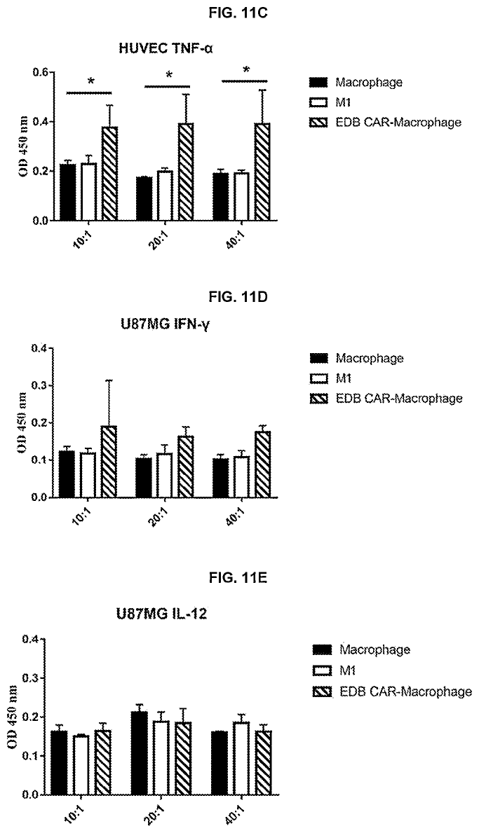

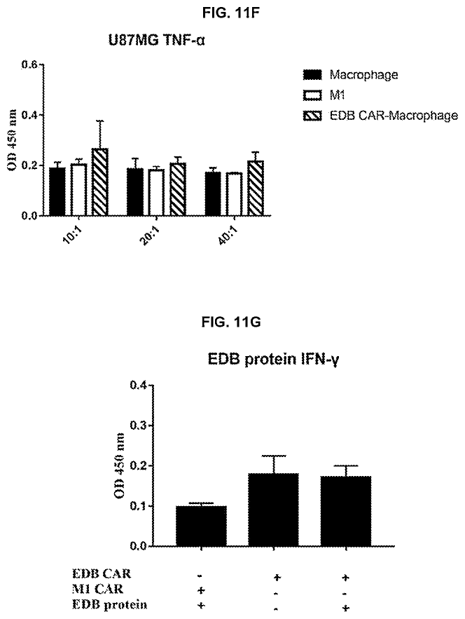

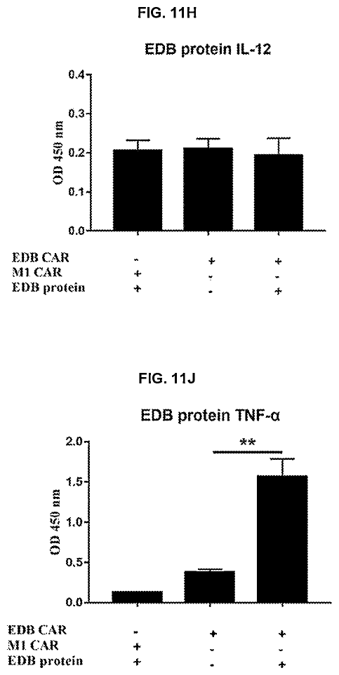

For cytokine induction of EDB-CAR-transduced monocytes/macrophages, target cells or EDB protein were mixed in effector-to-target ratios of 10, 20 and 40 in a 96-well U-bottom plate. After 24-hr culture, IFN-γ, TNF-α, and IL-12 expression were measured by ELISA (DAKEWE). See FIGS. 11A-11H, 11J.

A summary of the experimental procedure is provided below:

| 0 | 5 | 6 | 7 | 8 | 9 | 10 |
|---|---|---|---|---|---|---|
| 8.31 | 9.4 | 9.5 | 9.6 | 9.7 | 9.8 | 9.9 |

Day 1: Monocytes were isolated from peripheral blood and the ratio of CD14 + cells was detected by flow cytometry 10 ng/ml GM-CSF was added for 5 days.
Day 5: The adherent cells were digested and stained with trypan blue to calculate the cell viability.
Day 6: Virus transduction.
Day 7: Cell activity was observed.
Day 8: The positive rate of car was detected by flow cytometry.
Day 9: Incubated with target cells or EDB antigen.
Day 10: The expression of cytokines was detected by ELISA.

Earlier observation on the cytotoxicity of EDB-CAR T cells seemed to suggest that killing was not proportional to the expression levels of the EDB-fibronectin on the target cells. See Example 4. Further, EDB-CALR T cells secreted TNFα when co-cultured with target cells.

TNFα possesses cytotoxicity function. This example shows that the EDB-CAR-expressing monocytes/macrophages also secrete TNFα. See FIGS. 11C and 11J. It is known that interferon-γ and TNFα are both strong stimulators of immune cells and inhibitors of cancer cells. Thus it is believed that the subject EDB-CAR modified immune cells can be used to deliver cytokines such as IFN-7 and/or TNFα to cancer tissues to effect EDB-CAR-mediated killing.

### Example 8: Additional EDB-CAR Expressed in T Cells

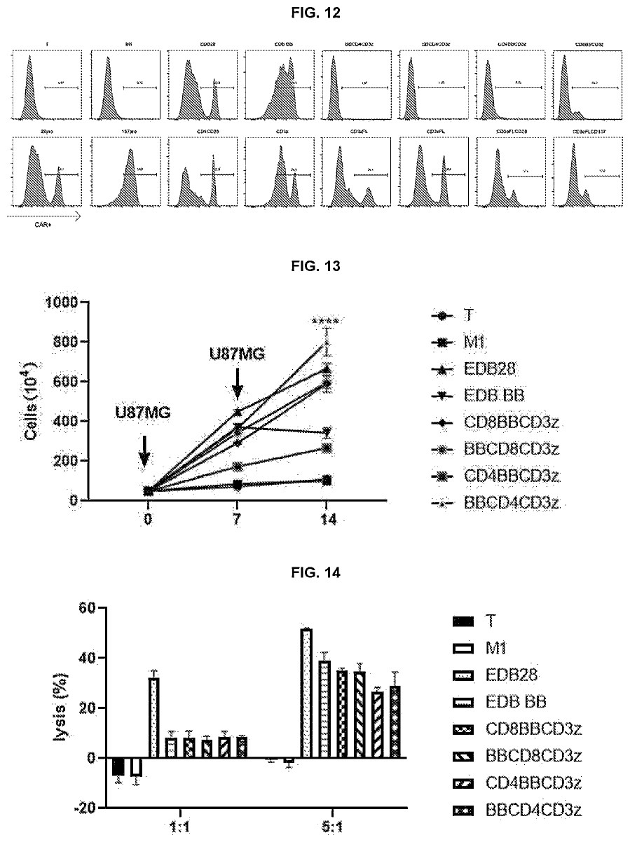

The CD4 and CD8 intracellular domains had been suggested to interact with the lymphocyte-specific protein tyrosine kinase (Lck) involved in T cells signaling. Intracellular domains of CD4ic and CD8ic were introduced into the second generation EDB CAR (SEQ. ID NO 1, 3), to generate EDB CARs with Lck recruitment potential (SEQ. ID NO 4, 5, 6, 7, 8). The expression test of CD4ic and CD8ic EDB CAR were carried out as described in Example 1 (See FIG. 12). The sequence IDs corresponding to the tested EDB CAR can be found in Table 1. Negative control for expression are represented by untransduced T cells (T) or T cells transduced with empty vector (M1). Expression for EDB CARs named as BBCD4CD3z and BBCD8CD3z (Sequence ID 4, 5) were barely detectable (FIG. 12) in this example. However, proliferative activities upon antigen induction and cytotoxicity towards cancer cells for the BBCD4CD3z and BBCD8CD3z CAR T cells are comparable to other second generation EDB CAR T cells (See Example 9, 10), suggesting that the BBCD4CD3z and BBCD8CD3z CAR T cells possess potent biological activities.

### Example 9: EDB-CAR T Cells Activated by Cancer Cells

Unlike transmembrane protein target, EDB CAR T cells target soluble EDB protein or EDB-containing fibronectin embedded in the extracellular matrix, therefore it is unclear whether antigen stimulation of EDB CAR T cells is sufficient in driving proliferation and cytotoxic activities. We tested 2^{nd} generation EDB CARs (defined as such for a single chimeric antigen receptor containing both primary signal and costimulatory signals), as well as CD4ic- and CD8ic-containing EDB CARs for proliferative activities upon antigen stimulation. Transduced T cells were stimulated with U87MG cells at the first and seventh days of this assay, in an effector to target ratio 5 to 1 in RPMI 1640 with 10% heat-inactivated FBS. Cells isolated at different time points were counted. As shown in FIG. 13, U87MG stimulated robust growth of transduced T cells, including the low expressor BBCD4CD3z and BBCD8CD3z (Sequence ID 4, 5) CAR T cells. To be noted is that transduced EDB CAR T cells in proliferation and cytotoxicity assays were adjusted to about 20% of total throughout this invention.

### Example 10: In Vitro Cytotoxicity of Various EDB-CAR T Cells to Cancer Cells

EDB CARs with both primary signal and costimulatory signals were tested for inducing cytotoxic activities of transduced T cells. Human primary T cells were transduced with the indicated lentiviral vectors and incubated with U87MG cells at the various E:T ratios for 24 hours. Cell lysis was determined using an LDH assay. Data are representative of three independent experiments (FIG. 14). 2^{nd} generation EDB CARs (defined as such for containing both primary signal and costimulatory signals), as well as CD4ic- and CD8ic-containing EDB CARs all demonstrated significant cytotoxic activities towards U87MG cancer cells. EDB28 CAR T cells registered highest in vitro cytotoxicity to the U87MG cancer cells. Similar to the observation in Example 9, the low expressor BBCD4CD3z and BBCD8CD3z (Sequence ID 4, 5) CAR T cells demonstrated cytotoxicity levels similar to the high expressor EDB CAR T cells.

### Example 11: Bi-Specific Molecule/EDB-CAR T Cells Activated by Cancer Cells

One aspect of the invention provides anti-EDB ScFv fused to anti-CD3ε ScFv, thus forming a bi-specific molecule, which can be used alone or in combination with a chimeric antigen receptor consisting of anti-EDB fibronectin ScFv fused to CD28, OX40, or CD137 proteins. In addition, bi-specific anti-EDB ScFv fused to anti-CD3ε ScFv could be secreted by modified T cells. In yet another application, bispecific anti-EDB ScFv fused to anti-CD3ε ScFv can be produced and used alone or in combination therapies. Bi-specific binders interact with both EDB antigen and the CD3ε on the T cells, thus activate the T cell proliferation and cytotoxicity.

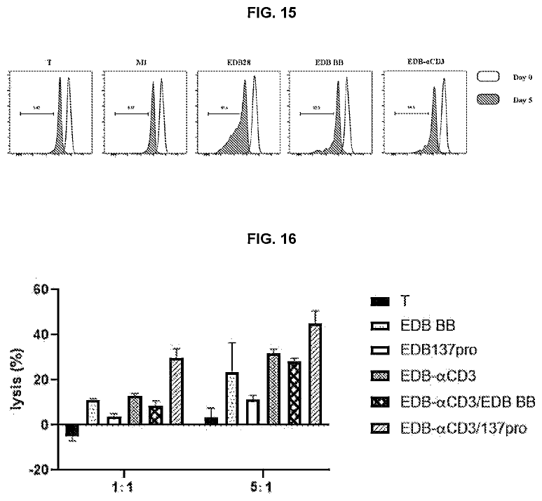

In this example, T cells were transduced with the bispecific binder EDB-αCD3 (Sequence ID NO: 18). Transduced T cells were labeled with CellTrace (Invitrogen, C34564) and stimulated with U87MG cells in the 1:5 effector to target ratio. All cells were grown in RPMI 1640 supplied with 10% heat-inactivated FBS. At different time points cells were analyzed by flow cytometry with a 630-nm excitation source (FIG. 15). Trailing fluorescence (bracketed area) relative to the main peak indicate proliferative activities of the T cells. Numbers above the bracket are the percentage of the total area, representing the proliferative portion of the T cells. This example demonstrates that bi-specific EDB-αCD3 alone is capable of stimulating T cells for proliferation.

### Example 12: Cytotoxicity of Bi-Specific Molecule/EDB-CAR T Cells to Cancer Cells

In vitro cytotoxic activities of the bi-specific EDB-αCD3 (Sequence ID NO: 18) molecule alone or transduced together with EDB-targeting CAR were tested using U87MG cells (FIG. 16). Primary human T cells transduced with the indicated molecules were incubated with U87MG cells at the various E:T ratios for 24 hours. Cell lysis was determined using an LDH assay. Data are representative of three independent experiments. Bi-specific EDB-αCD3 (Sequence ID NO: 18) combined with 137pro (Sequence ID NO: 10) seemingly possess synergistic cytotoxicity. Note that in this combination lentiviral vectors used to transduce the T cells were decreased to half, yet the cytotoxic activities were comparable to that for the 2^{nd} generation EDB CAR.

### Example 13: “Trans” EDB-CAR T Cells Activated by Cancer Cells

In the native TCR complex, CD3ζ intracellular domain containing the ITAMs is proximal to the plasma membrane. In most so-called 2^{nd} generation CAR, CD3 intracellular domain are fused to the co-stimulatory signals from CD28, 4-1BB or OX40, thus placing the ITAMs at a membrane-distal position. It has been reported that the CD3ζ phosphorylation is affected by its membrane proximity, while the distal position of the ITIMs impacted the phosphorylation. Thus EDB CAR were generated so that the EDB-specific ScFv were fused to the CD3ζ and CD28 extracellular domain. Similarly, EDB-specific ScFv can be fused to extracellular domain, transmembrane (TM) domain and intracellular domains of a membrane protein selected from CD3ε, CD3γ, CD3δ, CD4, CD8, OX40, CD28 or CD137, with or without linker regions connecting to the EDB-specific ScFv (For example SEQ ID NO: 9, 10, 11, 13, 14, 15). The extracellular domain and transmembrane domain may be derived from other proteins, e.g. CD8 (For example SEQ ID NO 12).

The anti-EDB fibronectin ScFv fused to full-length CD3ε, CD3γ, CD3δ or CD3ζ protein, the anti-EDB ScFv can be incorporated into the cellular T-cell receptor (TCR) complex, and clustering of the TCR complex is possible through binding of EDB cancer antigens by the anti-EDB fibronectin ScFv fused to part or full-length of CD3ε, CD3γ, CD3δ or CD3ζ proteins. In addition, the costimulatory signals can be provided by a separate polypeptide anti-EDB ScFv fused to the CD4, CD8, CD28, OX40, or CD137 protein. We define this type of fusion molecules as “trans” version of CAR as the co-stimulatory signaling domains are provided in separate polypeptide. The trans CAR contrast to the traditional, “cis” version of the CAR structures in which the co-stimulatory domain are in one single polypeptide chain.

“Trans” EDB CARs were generated with two polypeptide chains, in which one ScFv fused to CD3ε or CD3ζ for primary T cell activation signal, and another fused to CD137 or CD28 co-stimulatory domain. The ITAM domains of CD3ζ or co-stimulatory domains are expected to be proximal to the plasma membrane, thus in a format most similar to the native structures. Other molecules involved in TCR signaling such as LCK are anchored to the plasma membrane, therefore a “trans” CAR format might be more optimal for the formation of the signaling complex involving LCK. Any EDB CARs with primary stimulation signal (for example SEQ. ID NO: 11, 12, 13, 14, or 15) can be combined with EDB CARs with co-stimulatory signal (for examples SEQ. ID NO: 8, 9, 10) to generate a “trans” EDB CAR. In the current example T cells were transduced with anti EDB ScFv fused to CD3ζ (Sequence ID NO: 12), alone or together with EDB CAR molecules with co-stimulatory signals (Sequence ID NO: 9, 10, or 8, respectively).

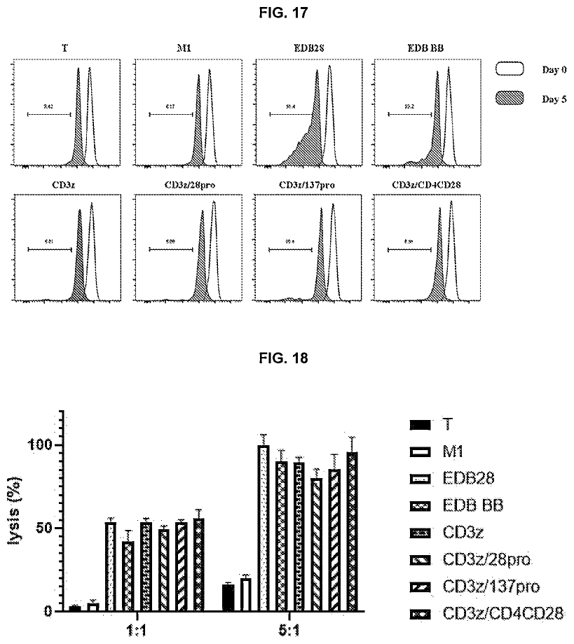

The expression test of “trans” EDB CAR were carried out as described in Example 1 (See FIG. 12). Transduced T cells were labeled with CellTrace (Invitrogen, C34564) and stimulated with U87MG cells in the 1:5 effector to target ratio. All cells were grown in RPMI 1640 supplied with 10% heat-inactivated FBS. At different time points cells were analyzed by flow cytometry with a 630-nm excitation source (FIG. 17). Trailing fluorescence (bracketed area) relative to the main peak indicate proliferative activities of the T cells. Numbers above the bracket are the percentage of the total area, representing the proliferative portion of the T cells.

### Example 14: Cytotoxicity of “Trans” EDB-CAR T Cells to Cancer Cells

“Trans” EDB CARs comprise of primary signal and costimulatory signals born on two separate polypeptide chains. To generate “trans” EDB CAR T cells, T cells were transduced with anti EDB ScFv fused to CD3ζ (Sequence ID NO: 12), alone or together with EDB CAR molecules with co-stimulatory signals (Sequence ID NO: 9, 10, or 8, respectively). Induction of the cytotoxic activities of transduced T cells were tested by incubation with target U87MG cells at various E:T ratios for 24 hours. Cell lysis was determined using an LDH assay. Data are representative of three independent experiments (FIG. 18). Note that in the “trans” format lentiviral vectors used to transduce the T cells were decreased to half, yet the cytotoxic activities of the “trans” EDB CAR T cells were comparable to that for the 2^{nd} generation EDB CAR or the 1^{st} generation CD3z CAR.

### Example 15: “Hijack” Molecule/EDB-CAR T Cells Activated by Cancer Cells

In the current invention chimeric antigen receptors were created with full length CD3ε, CD3ζ protein, with or without linker regions connecting to the EDB fibronectin binding domain. A typical TCR complex contain TCRα, TCRβ, CD3γ, CD3δ, CD3ζ, and CD3ε subunits. Protein fused to full-length CD3ε or CD3ζ are expected to be incorporated into the cellular T-cell receptor (TCR) complex, thus the chimeric TCRs that incorporated chimeric EDB CAR receptors can bind to cancer antigen EDB in a fashion independent of the MHC I complexes. In contrast to traditional CAR structures, CARs that complex with TCR are “hijack” CARs due to the acquisition of TCR signaling functions, with the benefit of the antigen specificity of the antigen-specific ScFv, and T cell activation by the chimeric TCR upon binding of tumor antigens. Any primary T cell with a chimeric TCR can be activated by the presence of the cancer antigen independent of MHC complexes. In brief, in the past the CAR structures were considered as standalone molecules. In the current invention the “hijack” EDB CAR are receptors engineered to be incorporated into the TCR complexes. Engaging EDB antigen by the chimeric “hijack” EDB CAR might trigger TCR activation, thus leading to proliferation and cytotoxicity of the EDB CAR T cells.

While “hijack” EDB CAR can provide the primary T cell stimulatory signals, the costimulatory signals can be provided by a separate polypeptide with EDB-specific ScFv fused to T cell activation domain of CD4, CD8, CD28, OX40, or CD137 protein. Such a format of EDB CAR can be considered as “trans” as well.

In this example the anti EDB ScFv were fused to CD3ε or CD3ζ full-length proteins, with or without linker sequences (Sequence ID NO: 11, 13, 14, 15). The CD3ε or CD3ζ polypeptide fragments used in this example contain sufficient structural information to be incorporated into the TCR complex. Indeed, some of the “hijack” EDB CAR (SEQ ID NO: 11, 13, 14, 15) were used as in a “trans” EDB CAR format as well. In this experiment, T cells were transduced with anti EDB ScFv fused to CD3ζ (Sequence ID NO: 11), or with anti EDB ScFv fused to CD3ε with 15 amino acid linkers (Sequence ID NO: 15), alone or together with EDB CAR molecules with co-stimulatory signals (Sequence ID NO: 9, 10, or 8, respectively). The expression test of “hijack” EDB CAR were carried out as described in Example 1 (See FIG. 12). Transduced T cells were labeled with CellTrace (Invitrogen, C34564) and stimulated with U87MG cells in the 1:5 effector to target ratio. All cells were grown in RPMI 1640 supplied with 10% heat-inactivated FBS. At different time points cells were analyzed by flow cytometry with a 630-nm excitation source (FIG. 19). Trailing fluorescence (bracketed area) relative to the main peak indicate proliferative activities of the T cells. Numbers above the bracket are the percentage of the total area, representing the proliferative portion of the T cells. Enhanced proliferation was seen with “hijack” EDB CAR in combination with the CD28 co-stimulatory domain carried in trans by a separate EDB CAR polypeptide chain.

### Example 16: Cytotoxicity of “Hijack” EDB-CAR T Cells to Cancer Cells

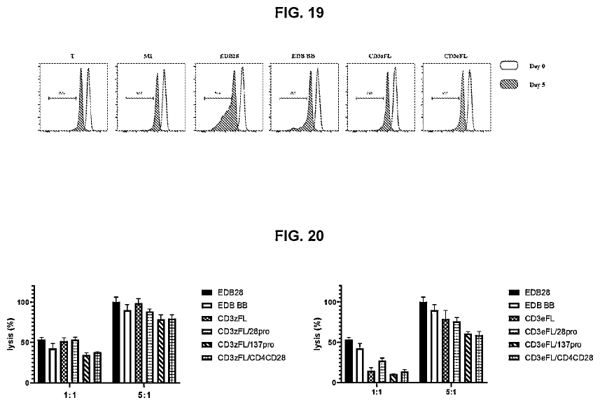

“Hijack” EDB CARs comprise of anti EDB ScFv fused to full-length CD3ζ or CD3ε proteins, with or without the co-stimulatory molecules born on a separate polypeptide chain. To generate “hijack” EDB CAR T cells, T cells were transduced with anti EDB ScFv fused to CD3ζ full-length (Sequence ID NO: 11), alone or together with EDB CAR molecules with co-stimulatory signals (Sequence ID NO: 9, 10, or 8, respectively). In another version of “hijack” EDB CAR T cells, T cells were transduced with anti EDB ScFv fused to CD3ε full-length (Sequence ID NO: 13, 14, or 15), alone or together with EDB CAR molecules with co-stimulatory signals (Sequence ID NO: 9, 10, or 8, respectively). Induction of the cytotoxic activities of transduced T cells were tested by incubation with target U87MG cells at various E:T ratios for 24 hours. Cell lysis was determined using an LDH assay. Data are representative of three independent experiments (FIG. 20). Note that when “hijack” EDB CAR were combined with co-stimulatory EDB CAR molecules, lentiviral vectors used to transduce the T cells were reduced to half.

### Example 17: “Hijack Plus” EDB-CAR T Cells Activated by Cancer Cells

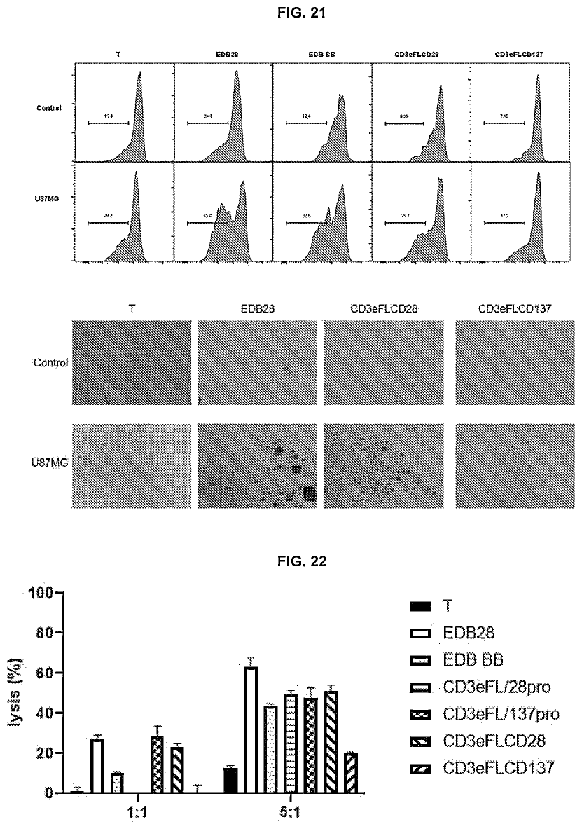

In the “trans” format of the “hijack” CARs, the anti-EDB fibronectin ScFv could be fused to full-length CD3ε, CD3γ, CD3δ or CD3ζ (hijack CAR), or fused to part or full length of CD4, CD8, CD28, OX40, or CD137 to provide the co-stimulatory signaling domains in a separate polypeptide, i.e. in trans. While the “Hijack” EDB CAR are receptors engineered to be incorporated into the TCR complexes, per se they lack co-stimulatory domains required for enhanced proliferation. Particularly for a target like EDB, it is unclear whether the separation of the primary and co-stimulatory signals onto separate polypeptide chains leads to most optimal activation of the T cells. To address this question, a “cis” format for the primary and co-stimulatory signals was generated, i.e. a co-stimulatory domain was incorporated into a “hijack” EDB CAR (SEQ. ID NO: 16, 17). In this scenario the chimeric TCR recognize the EDB antigen, provides primary survival signals through TCR activation, and enhances the proliferation and cytotoxic function of T cells through the attached co-stimulatory domain. To engineer a “hijack” EDB CAR with a co-stimulatory domain, the EDB CD3εFL (Sequence ID NO: 15) were attached at its C-terminus the co-stimulatory domain of CD28 or that of CD137, resulting in “hijack plus” EDB CAR constructs (Sequence ID NO: 16, 17). Indeed, for “hijack plus” CARs the anti-EDB fibronectin ScFv can be fused to full-length CD3ε, CD3γ, CD3δ or CD3ζ followed by part or full length of CD4, CD8, CD28, OX40, or CD137 to provide the co-stimulatory signaling domains in the same polypeptide chain. To test the activation of the “hijack plus” EDB CAR T cells by exposure to antigen, T cells were transduced with anti “hijack plus EDB CAR (Sequence ID NO: 16 or 17, respectively). The expression test of “hijack plus” EDB CAR were carried out as described in Example 1 (See FIG. 12). Transduced T cells were labeled with CellTrace (Invitrogen, C34564) and stimulated with U87MG cells in the 1:5 effector to target ratio. All cells were grown in RPMI 1640 supplied with 10% heat-inactivated FBS. At different time points cells were analyzed by flow cytometry with a 630-nm excitation source (FIG. 21, upper panel). Trailing fluorescence (bracketed area) relative to the main peak indicate proliferative activities of the T cells. Numbers above the bracket are the percentage of the total area, representing the proliferative portion of the T cells. Proliferation activation was demonstrated with “hijack plus” EDB CAR T cells in contrast to controls when no antigen was present.

The bottom panel of the FIG. 21 depicts the morphology of “hijack plus” EDB CAR T cells after exposure to U87MG cells. EDB28 CAR T (Sequence ID NO 3) cells demonstrates the highest aggregation, mostly due the surface adhesion proteins upon T cells activation. The “hijack plus” CD3eFLCD28 CAR T (Sequence ID NO. 16) cells proliferated and aggregated significantly upon the induction by U87MG cells.

### Example 18: Cytotoxicity of “Hijack Plus” EDB-CAR T Cells to Cancer Cells

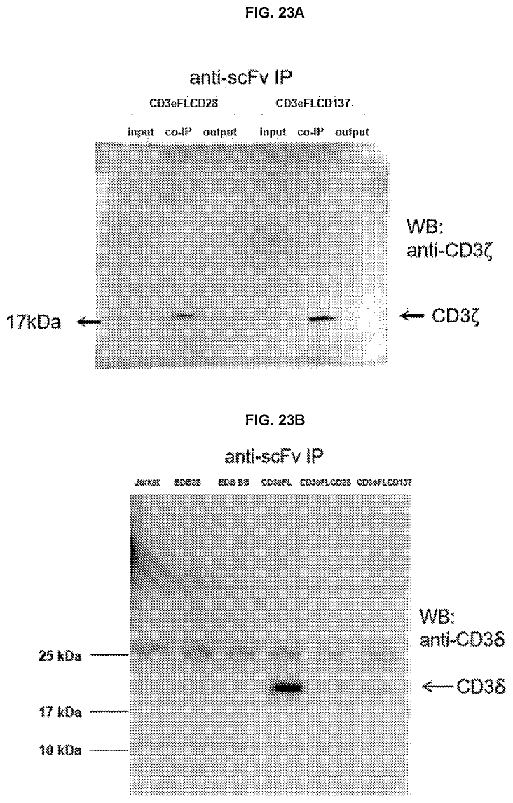

“Hijack plus” EDB CARs comprise of anti EDB ScFv fused to full-length CD3ζ or CD3ε proteins with the co-stimulatory molecules born on the same polypeptide chain. Thus the “hijack plus” EDB CAR primary and co-stimulatory signals are in a cis format. To test whether this cis format could still activated cytotoxicity of the transduced T cells, “Hijack plus” EDB CAR T cells were generated, and induction of the cytotoxic activities of transduced T cells were tested by incubation with target U87MG cells at various E:T ratios for 24 hours. Cell lysis was determined using an LDH assay. Data are representative of three independent experiments (FIG. 22). The “hijack plus” EDB CAR with CD28 co-stimulatory signal (SEQ. ID NO 16) was active in cytotoxic activities. Note that the “hijack plus” EDB CAR with CD137 co-stimulatory molecule (SEQ. ID NO. 17) demonstrated mild yet significant cytotoxic activities.

### Example 19: Incorporation of “Hijack Plus EDB CAR Cells into TCR Complex

Although “hijack plus” EDB CARs contain CD3ζ or CD3ε polypeptide fragments with structural information for incorporation into the TCR complex, it is unclear whether the incorporation is inhibited by fusion to the CD28 or CD137 co-stimulatory domains. In this example, T cells transduced with “hijack plus” EDB CARs were solubilized by the NP-40 detergent, and immune precipitated by using biotin-labeled polyclonal goat anti-human-IgG F(ab′)₂ fragment antibodies (Jackson Immunoresearch, Cat #109-066-097). The immune precipitates were analyzed by Western blotting using CD3ζ-specific antibody (FIG. 23A), or by Western blotting using CD36-specific antibody (FIG. 23B). For CD3ζ a band of about 17 kDa complexed with “hijack plus” EDB CARs (SEQ. ID NO 16, 17) was detected (FIG. 23A), while for CD3δ a band of about 19 kDa complexed with “hijack plus” or “hijack” EDB CARs (SEQ. ID NO 15, 16, 17) was detected (FIG. 23B). This example demonstrates that the “hijack” or “hijack plus” EDB CARs (SEQ. ID NO 15, 16 or 17) were associated with the CD3δ and CD3ζ, suggesting proper incorporation of these EDB CARs into the TCR complex and the co-stimulatory domains fused to the C-terminus of CD3ζ or CD3ε does not interfere with the assembly of the “hijack plus” EDB CARs.

### Example 20: In Vivo Reduction of Tumor and Inhibition of Tumor Growth

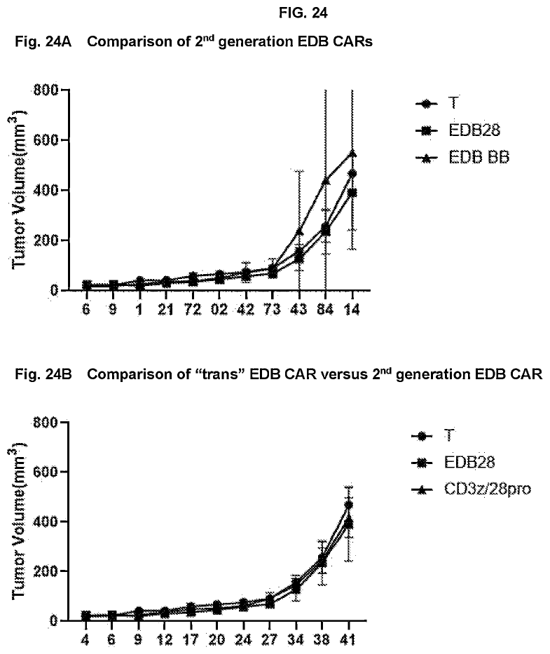

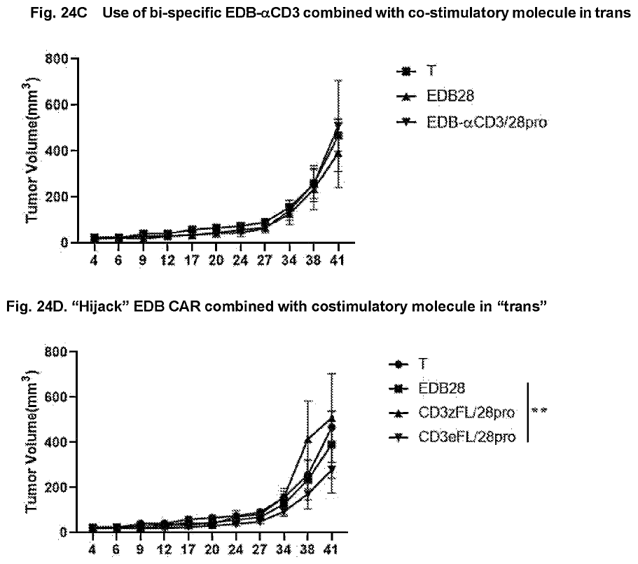

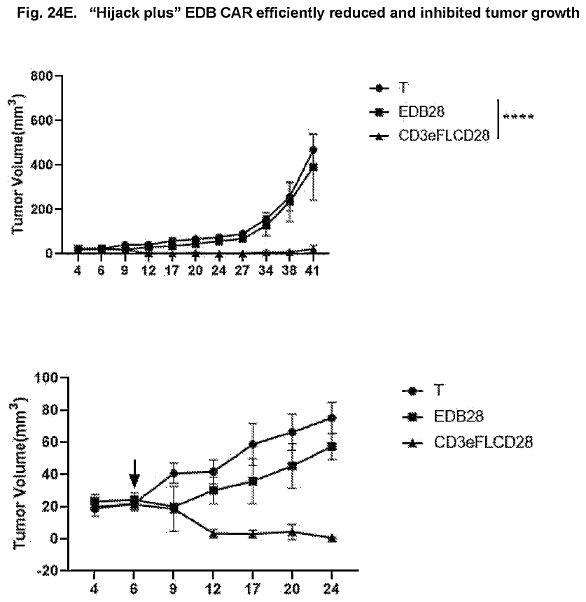

In the current invention a series of EDB CAR was constructed, and all of which demonstrated in vitro, antigen-specific activation of the T cells and induction of cytotoxic activities. However, it is unclear whether any of the engineered EDB CAR T cells are capable of penetrating the tumor tissue and kill the tumor cells. Published work (Wagner et al., 2021, DOI: 10.1158/2326-6066.CIR-20-0280) showed use of a 2^{nd} generation EDB CAR T cells and demonstrated only mild inhibition of tumor growth, and no reduction of established tumor tissues. This examples aims at analysis of in vivo efficacy of the EDB CARs of the current invention. Immune compromised NCG mice was used to establish the U87MG tumor model, and approximately 1 million U87MG cells were injected subcutaneously to establish tumor on the dorsal side of 6-week old NCG mice. Once the tumors were palpable and the tumor sizes measured to approximately 20-50 cubic millimeters, mice were sorted into groups (5/group). 5 million transduced EDB CAR T cells of various kind were infused by tail-vein injection into the mice. As reported by literature, 2^{nd} generation EDB CAR demonstrated little or no inhibition of tumor growth (FIG. 24. A). “Trans” or Bi-specific EDB CAR combined with costimulatory signals did not demonstrate significant inhibition of tumor growth (FIGS. 24. B and C). “Hijack” EDB CAR T cells demonstrated significant but only mild inhibition of tumor growth (FIG. 24. D). The most efficacious inhibition has been observed with “hijack plus” EDB CAR T cells, and the tumor growth was retarded for prolonged period of time (FIG. 24. E upper panel). It is interesting to note that the established tumor was reduced to barely detectable in the early phase of treatment (FIG. 24. E bottom panel), suggesting that “hijack plus” EDB CAR T cells were able to penetrate the tumor tissue and lyse the tumor cells, achieving almost complete regression. The tumor tissue were not palpable 10 days after infusions of the “hijack plus” EDB CAR T cells. Thus, the cytotoxic activities of the “hijack plus” EDB CAR T cells were not affected by the immune suppressive microenvironment, i.e. the activated T cells were not inhibited by immune suppression of the tumor tissue and was able to lyse the tumor cells, suggesting that the “hijack plus” EDB CAR T cells could be used as therapeutic agent for cancer treatment even for tumor with strong immune suppressive microenvironment.

The experimental findings from the in vivo studies suggest that only “hijack plus” EDB CAR were efficacious in significant tumor regression and tumor growth inhibitions, a finding consistent with the earlier observation that 2^{nd} generation EDB CARs with CD3ζ and CD28 intracellular domains performed poorly in controlling the tumor growth. The deficiency of the 2^{nd} generation EDB CAR in vivo has yet to be elucidated, and is most likely due to the particular characteristics of EDB antigen itself. EDB-containing fibronectin are constituents of extracellular matrix (ECM), and its structural complexity in the tumor tissues indicate multiple presences including ECM, interstitial deposits, and perivascular locations. Neither of the EDB-containing fibronectin forms were integral membrane proteins and lack lateral fluidity of a typical membrane protein in vivo. Therefore it is possible that in vivo the 2^{nd} generation EDB CAR with CD3ζ and CD28 intracellular domains are insufficient in forming clustered structures and lack proper enhancement of T cell activation and proliferations.

In contrast both in vitro and in vivo cytotoxicity were demonstrated for “hijack plus” EDB CAR T cells. Our findings at minimum show that “hijack plus” EDB CAR T cells were activated and cytotoxic in vivo. The chimeric TCR receptors were capable of activating T cell cytotoxicity and clear large tumor burdens in vivo. The mechanisms for the activation of the chimeric TCR activation remain unclear, but require TCR activation and the presence of a co-stimulatory signaling domain in cis format. Combined with the finding that 2^{nd} generation EDB CAR with CD3ζ were insufficient in controlling the tumor growth in vivo, this example suggests that the “hijack” EDB CARs also signal through component(s) in the TCR complexes in addition to the CD3ζ ITAMs, such as through CD3δ and/or CD3γ. In addition, the activation of chimeric “hijack plus” TCR complex must contain both primary and co-stimulatory signals, suggesting that the proximity of the signalosomes for the primary and co-stimulatory signals are critical for in vivo activation of EDB-targeting chimeric antigen receptors.

Sequence listing in FASTA format:

>EDB137ic
MYRMQLLSCIALSLALVTNSEVQLLESGGGLVQPGGSLRLSCAASGFTFSSESMSWVRQAPG
KGLEWVSSISGSSGTTYYADSVKGRFTISRDNSKNTLYLQMNSLRAEDTAVYYCAKPFPYFD
YWGQGTLVTVSSGDGSSGGSGGASEIVLTQSPGTLSLSPGERATLSCRASQSVSSSFLAWYQ
QKPGQAPRLLIYYASSRATGIPDRFSGSGSGTDFTLTISRLEPEDFAVYYCQQTGRIPPTFG
QGTKVEIKAKPTTTPAPRPPTPAPTIASQPLSLRPEACRPAAGGAVHTRGLDFACDIYIWAP
LAGTCGVLLLSLVITLYKRGRKKLLYIFKQPEMRPVQTTQEEDGCSCRFPEEEEGGCELRVK
FSRSADAPAYQQGQNQLYNELNLGRREEYDVLDKRRGRDPEMGGKPQRRKNPQEGLYNELQK
DKMAEAYSEIGMKGERRRGKGHDGLYQGLSTATKDTYDALHMQALPPR (SEQ ID NO: 1)
>EDB137ic nucleotide sequence
ATGTACAGAATGCAGCTGCTGTCCTGCATCGCCCTGAGCCTGGCCCTGGTGACCAATAGCGA
GGTGCAACTCCTGGAGTCCGGCGGAGGCCTGGTCCAACCTGGAGGAAGCCTGAGGCTGAGCT
GTGCCGCCAGCGGCTTCACCTTTTCCAGCTTCTCCATGAGCTGGGTGAGACAGGCCCCCGGC
AAAGGCCTGGAGTGGGTGTCCAGCATCTCCGGCAGCTCCGGCACCACCTACTATGCTGATTC
CGTGAAGGGCAGGTTCACCATCTCCAGGGACAACAGCAAGAACACACTGTACCTCCAAATGA
ACTCCCTGAGGGCCGAAGACACCGCCGTGTACTACTGCGCCAAGCCCTTTCCCTATTTCGAC
TATTGGGGCCAGGGCACACTGGTCACCGTGAGCTCCGGCGATGGAAGCAGCGGAGGAAGCGG
AGGCGCTAGCGAAATCGTGCTGACCCAGAGCCCTGGCACACTGTCCCTGAGCCCTGGAGAAA
GAGCCACCCTGAGCTGTAGGGCCTCCCAGAGCGTGAGCAGCAGCTTCCTGGCCTGGTACCAA
CAGAAGCCCGGACAGGCCCCCAGGCTGCTGATCTACTATGCCTCCTCCAGGGCCACAGGCAT
CCCCGACAGGTTCTCCGGCTCCGGTTCTGGCACCGATTTTACCCTGACCATCTCCAGGCTGG
AGCCCGAAGACTTCGCCGTGTATTACTGCCAGCAGACCGGACGTATTCCTCCTACCTTTGGC
CAGGGCACCAAGGTGGAGATCAAAGCCAAGCCCACCACCACACCTGCCCCTAGACCCCCTAC
ACCTGCCCCCACAATCGCTTCCCAGCCTCTGTCCCTGAGGCCTGAGGCTTGTAGGCCTGCCG
CTGGAGGAGCTGTGCACACCAGAGGCCTCGACTTCGCCTGCGACATCTATATCTGGGCTCCT
CTGGCCGGCACCTGTGGAGTCCTCCTGCTGAGCCTGGTGATCACACTGTACAAGAGAGGCAG
GAAGAAGCTGCTGTACATCTTCAAGCAACCCTTCATGAGGCCTGTGCAGACCACCCAGGAAG
AAGATGGCTGCAGCTGCAGGTTCCCTGAGGAAGAAGAGGGCGGATGCGAGCTGAGAGTGAAG
TTCAGCAGGTCCGCCGATGCCCCTGCCTATCAGCAGGGCCAGAACCAGCTGTACAACGAACT
CAACCTGGGCAGGAGGGAGGAGTACGACGTCCTCGACAAGAGGAGAGGCAGGGACCCCGAGA
TGGGAGGCAAGCCTCAGAGGAGGAAGAACCCTCAAGAGGGACTGTACAACGAGCTGCAGAAG
GACAAGATGGCCGAGGCCTACTCCGAGATCGGCATGAAGGGCGAGAGAAGAAGAGGCAAGGG
CCATGATGGCCTCTACCAGGGCCTGAGCACCGCCACCAAGGACACATACGATGCCCTGCATA
TGCAGGCCCTCCCCCCTAGGTGA (SEQ ID NO: 2)
>EDB28ic
MYRMQLLSCIALSLALVTNSEVQLLESGGGLVQPGGSLRLSCAASGFTFSSFSMSWVRQAPG
KGLEWVSSISGSSGTTYYADSVKGRFTISRDNSKNTLYLQMNSLRAEDTAVYYCAKPFPYFD
YWGQGTLVTVSSGDGSSGGSGGASEIVLTQSPGTLSLSPGERATLSCRASQSVSSSFLAWYQ
QKPGQAPRLLIYYASSRATGIPDRFSGSGSGTDFTLTISRLEPEDFAVYYCQQTGRIPPTFG
QGTKVEIKAKPTTTPAPRPPTPAPTIASQPLSLRPEACRPAAGGAVHTRGLDFACDIYIWAP
LAGTCGVLLLSLVITLYRSKRSRLLHSDYMNMTPRRPGPTRKHYQPYAPPRDFAAYRSRVKF
SRSADAPAYQQGQNQLYNELNLGRREEYDVLDKRRGRDPEMGGKPQRRKNPQEGLYNELQKD
KMAEAYSEIGMKGERRRGKGHDGLYQGLSTATKDTYDALHMQALPPR (SEQ ID NO: 3)
>EDB 41BB-CD4ic-CD3z
MYRMQLLSCIALSLALVTNSEVQLLESGGGLVQPGGSLRLSCAASGFTFSSFSMSWVRQAPG
KGLEWVSSISGSSGTTYYADSVKGRFTISRDNSKNTLYLQMNSLRAEDTAVYYCAKPFPYFD
YWGQGTLVTVSSGDGSSGGSGGASEIVLTQSPGTLSLSPGERATLSCRASQSVSSSFLAWYQ
QKPGQAPRLLIYYASSRATGIPDRFSGSGSGTDFTLTISRLEPEDFAVYYCQQTGRIPPTFG
QGTKVEIKAKPTTTPAPRPPTPAPTIASQPLSLRPEACRPAAGGAVHTRGLDFACDIYIWAP
LAGTCGVLLLSLVITLYKRGRKKLLYIFKQPFMRPVQTTQEEDGCSCRFPEEEEGGCELCVR
CRHRRRQAERMSQIKRLLSEKKTCQCPHRFQKTCSPIRVKFSRSADAPAYQQGQNQLYNELN
LGRREEYDVLDKRRGRDPEMGGKPQRRKNPQEGLYNELQKDKMAEAYSEIGMKGERRRGKGH
DGLYQGLSTATKDTYDALHMQALPPR (SEQ ID NO: 4)
>EDB 41BB-CD8ic-CD3Z
MYRMQLLSCIALSLALVINSEVQLLESGGGLVQPGGSLRLSCAASGFTFSSFSMSWVRQAPG
KGLEWVSSISGSSGTTYYADSVKGRFTISRDNSKNTLYLQMNSLRAEDTAVYYCAKPFPYFD
YWGQGTLVTVSSGDGSSGGSGGASEIVLTQSPGTLSLSPGERATLSCRASQSVSSSFLAWYQ
QKPGQAPRLLIYYASSRATGIPDRFSGSGSGTDFTLTISRLEPEDFAVYYCQQTGRIPPTFG
QGTKVEIKAKPTTTPAPRPPTPAPTIASQPLSLRPEACRPAAGGAVHTRGLDFACDIYIWAP
LAGTCGVLLLSLVITLYKRGRKKLLYIFKQPFMRPVQTTQEEDGCSCRFPEEEEGGCELLYC
NHRNRRRVCKCPRPVVKSGDKPSLSARYVRVKFSRSADAPAYQQGQNQLYNELNLGRREEYD
VLDKRRGRDPEMGGKPQRRKNPQEGLYNELQKDKMAEAYSEIGMKGERRRGKGHDGLYQGLS
TATKDTYDALHMQALPPR (SEQ ID NO: 5)
>EDB CD4ic-41BB-CD3Z
MYRMQLLSCIALSLALVINSEVQLLESGGGLVQPGGSLRLSCAASGFTFSSFSMSWVRQAPG
KGLEWVSSISGSSGTTYYADSVKGRFTISRDNSKNTLYLQMNSLRAEDTAVYYCAKPFPYFD
YWGQGTLVTVSSGDGSSGGSGGASEIVLTQSPGTLSLSPGERATLSCRASQSVSSSFLAWYQ
QKPGQAPRLLIYYASSRATGIPDRFSGSGSGTDFTLTISRLEPEDFAVYYCQQTGRIPPTFG
QGTKVEIKAKPTTTPAPRPPTPAPTIASQPLSLRPEACRPAAGGAVHTRGLDFACDIYIWAP
LAGTCGVLLLSLVITLYLYCNHRNRRRVCKCPRPVVKSGDKPSLSARYVKRGRKKLLYIFKQ
PFMRPVQTTQEEDGCSCRFPEEEEGGCELRVKFSRSADAPAYQQGQNQLYNELNLGRREEYD
VLDKRRGRDPEMGGKPQRRKNPQEGLYNELQKDKMAEAYSEIGMKGERRRGKGHDGLYQGLS
TATKDTYDALHMQALPPR (SEQ ID NO: 6)
>EDB CD8ic-BB-CD3z
MYRMQLLSCIALSLALVTNSEVQLLESGGGLVQPGGSLRLSCAASGFTFSSFSMSWVRQAPG
KGLEWVSSISGSSGTTYYADSVKGRFTISRDNSKNTLYLQMNSLRAEDTAVYYCAKPFPYFD
YWGQGTLVTVSSGDGSSGGSGGASEIVLTQSPGTLSLSPGERATLSCRASQSVSSSFLAWYQ
QKPGQAPRLLIYYASSRATGIPDRFSGSGSGTDFTLTISRLEPEDFAVYYCQQTGRIPPTFG
QGTKVEIKAKPTTTPAPRPPTPAPTIASQPLSLRPEACRPAAGGAVHTRGLDFACDIYIWAP
LAGTCGVLLLSLVITLYLYCNHRNRRRVCKCPRPVVKSGDKPSLSARYVKRGRKKLLYIFKQ
PFMRPVQTTQEEDGCSCRFPEEEEGGCELRVKFSRSADAPAYQQGQNQLYNELNLGRREEYD
VLDKRRGRDPEMGGKPQRRKNPQEGLYNELQKDKMAEAYSEIGMKGERRRGKGHDGLYQGLS
TATKDTYDALHMQALPPR (SEQ ID NO: 7)
>EDB CD4ic-CD28ic
MYRMQLLSCIALSLALVTNSEVQLLESGGGLVQPGGSLRLSCAASGFTFSSFSMSWVRQAPG
KGLEWVSSISGSSGTTYYADSVKGRFTISRDNSKNTLYLQMNSLRAEDTAVYYCAKPFPYFD
YWGQGTLVTVSSGDGSSGGSGGASEIVLTQSPGTLSLSPGERATLSCRASQSVSSSFLAWYQ
QKPGQAPRLLIYYASSRATGIPDRFSGSGSGTDFTLTISRLEPEDFAVYYCQQTGRIPPTFG
QGTKVEIKAKPTTTPAPRPPTPAPTIASQPLSLRPEACRPAAGGAVHTRGLDFACDIYIWAP
LAGTCGVLLLSLVITLYCVRCRHRRRQAERMSQIKRLLSEKKTCQCPHRFQKTCSPIRSKRS
RLLHSDYMNMTPRRPGPTRKHYQPYAPPRDFAAYRS (SEQ ID NO: 8)
>EDB CD28 aa138-220
MYRMQLLSCIALSLALVTNSEVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFPYFDYWGQGTLVTVSSGDGSSG
GSGGASEIVLTQSPGTLSLSPGERATLSCRASQSVSSSFLAWYQQKPGQAPRLLIYYASSRATGIPDRFSG
SGSGTDFTLTISRLEPEDFAVYYCQQTGRIPPTFGQGTKVEIKKHLCPSPLFPGPSKPFWVLVVVGGVLAC
YSLLVTVAFIIFWVRSKRSRLLHSDYMNMTPRRPGPTRKHYQPYAPPRDFAAYRS (SEQ ID NO: 9)
>EDB CD137 aa160-255
MYRMQLLSCIALSLALVTNSEVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFPYFDYWGQGTLVTVSSGDGSSG
GSGGASEIVLTQSPGTLSLSPGERATLSCRASQSVSSSFLAWYQQKPGQAPRLLIYYASSRATGIPDRFSG
SGSGTDFTLTISRLEPEDFAVYYCQQTGRIPPTFGQGTKVEIKPSPADLSPGASSVTPPAPAREPGHSPQI
ISFFLALTSTALLFLLFFLTLRFSVVKRGRKKLLYIFKQPFMRPVQTTQEEDGCSCRFPEEEEGGCEL
(SEQ ID NO: 10)
>EDB CD3zFL
MYRMQLLSCIALSLALVTNSEVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFPYFDYWGQGTLVTVSSGDGSSG
GSGGASEIVLTQSPGTLSLSPGERATLSCRASQSVSSSFLAWYQQKPGQAPRLLIYYASSRATGIPDRFSG
SGSGTDFTLTISRLEPEDFAVYYCQQTGRIPPTFGQGTKVEIKAKPTTTPAPRPPTPAPTIASQPLSLRPE
CARPAAGGAVHQSFGLLDPKLCYLLDGILFIYGVILTALFLRVKFSRSADAPAYQQGQNQLYNELNLGRRE
EYDVLDKRRGRDPEMGGKPQRRKNPQEGLYNELQKDKMAEAYSEIGMKGERRRGKGHDGLYQGLSTATKDT
YDALHMQALPPR (SEQ ID NO: 11)
>EDB CD3zic
MYRMQLLSCIALSLALVTNSEVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFPYFDYWGQGTLVTVSSGDGSSG
GSGGASEIVLTQSPGTLSLSPGERATLSCRASQSVSSSFLAWYQQKPGQAPRLLIYYASSRATGIPDRFSG
SGSGTDFTLTISRLEPEDFAVYYCQQTGRIPPTFGQGTKVEIKAKPTTTPAPRPPTPAPTIASQPLSLRPE
ACRPAAGGAVHTRGLDFACDIYIWAPLAGTCGVLLLSLVITLYRVKFSRSADAPAYQQGQNQLYNELNLGR
REEYDVLDKRRGRDPEMGGKPQRRKNPQEGLYNELQKDKMAEAYSEIGMKGERRRGKGHDGLYQGLSTATK
DTYDALHMQALPPR (SEQ ID NO: 12)
>EDB 5 aaCD3eFL
MYRMQLLSCIALSLALVTNSEVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFPYFDYWGQGTLVTVSSGDGSSG
GSGGASEIVLTQSPGTLSLSPGERATLSCRASQSVSSSFLAWYQQKPGQAPRLLIYYASSRATGIPDRFSG
SGSGTDFTLTISRLEPEDFAVYYCQQTGRIPPTFGQGTKVEIKGRASGDGNEEMGGITQTPYKVSISGTTV
ILTCPQYPGSEILWQHNDKNIGGDEDDKNIGSDEDHLSLKEFSELEQSGYYVCYPRGSKPEDANGYLYLRA
RVCENCMEMDVMSVATIVIVDICITGGLLLLVYYWSKNRKAKAKPVTRGAGAGGRQRGQNKERPPPVPNPD
YEPIRKGQRDLYSGLNQRRI (SEQ ID NO: 13)
>EDB 10 aalinker CD3eFL
MYRMQLLSCIALSLALVTNSEVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFPYFDYWGQGTLVTVSSGDGSSG
GSGGASEIVLTQSPGTLSLSPGERATLSCRASQSVSSSFLAWYQQKPGQAPRLLIYYASSRATGIPDRESG
SGSGTDFTLTISRLEPEDFAVYYCQQTGRIPPTFGQGTKVEIKGGGGSGGGGSDGNEEMGGITQTPYKVSI
SGTTVILTCPQYPGSEILWQHNDKNIGGDEDDKNIGSDEDHLSLKEFSELEQSGYYVCYPRGSKPEDANFY
LYLRARVCENCMEMDVMSVATIVIVDICITGGLLLLVYYWSKNRKAKAKPVTRGAGAGGRQRGQNKERPPP
VPNPDYEPIRKGQRDLYSGLNQRRI (SEQ ID NO: 14)
>EDB 15aalinker CD3eFL
MYRMQLLSCIALSLALVTNSEVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFPYFDYWGQGTLVTVSSGDGSSG
GSGGASEIVLTQSPGTLSLSPGERATLSCRASQSVSSSFLAWYQQKPGQAPRLLIYYASSRATGIPDRFSG
SGSGTDFTLTISRLEPEDFAVYYCQQTGRIPPTFGQGTKVEIKGGGGSGGGGSGGGGSDGNEEMGGITQTP
YKVSISGTTVILTCPQYPGSEILWQHNDKNIGGDEDDKNIGSDEDHLSLKEFSELEQSGYYVCYPRGSKPE
DANFYLYLRARVCENCMEMDVMSVATIVIVDICITGGLLLLVYYWSKNRKAKAKPVTRGAGAGGRQRGQNK
ERPPPVPNPDYEPIRKGQRDLYSGLNQRRI (SEQ ID NO: 15)
>EDB 15 aalinker CD3eFL CD28ic
MYRMQLLSCIALSLALVTNSEVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFPYFDYWGQGTLVTVSSGDGSSG
GSGGASEIVLTQSPGTLSLSPGERATLSCRASQSVSSSFLAWYQQKPGQAPRLLIYYASSRATGIPDRFSG
SGSGTDFTLTISRLEPEDFAVYYCQQTGRIPPTFGQGTKVEIKGGGGSGGGGSGGGGSDGNEEMGGITQTP
YKVSISGTTVILTCPQYPGSEILWQHNDKNIGGDEDDKNIGSDEDHLSLKEFSELEQSGYYVCYPRGSKPE
DANFYLYLRARVCENCMEMDVMSVATIVIVDICITGGLLLLVYYWSKNRKAKAKPVTRGAGAGGRQRGQNK
ERPPPVPNPDYEPIRKGQRDLYSGLNQRRIRSKRSRLLHSDYMNMTPRRPGPTRKHYQPYAPPRDFAAYRS
L
(SEQ ID NO: 16)
>EDB 15 aalinker CD3eFL CD137ic
MYRMQLLSCIALSLALVTNSEVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFPYFDYWGQGTLVTVSSGDGSSG
GSGGASEIVLTQSPGTLSLSPGERATLSCRASQSVSSSFLAWYQQKPGQAPRLLIYYASSRATGIPDRFSG
SGSGTDFTLTISRLEPEDFAVYYCQQTGRIPPTFGQGTKVEIKGGGGSGGGGSGGGGSDGNEEMGGITQTP
YKVSISGTTVILTCPQYPGSEILWQHNDKNIGGDEDDKNIGSDEDHLSLKEFSELEQSGYYVCYPRGSKPE
DANFYLYLRARVCENCMEMDVMSVATIVIVDICITGGLLLLVYYWSKNRKAKAKPVTRGAGAGGRQRGQNK
ERPPPVPNPDYEPIRKGQRDLYSGLNQRRIKRGRKKLLYIFKQPFMRPVQTTQEEDGCSCRFPEEEEGGCE
L (SEQ ID NO: 17)
>EDB-αCD3
MYRMQLLSCIALSLALVTNSEVQLLESGGGLVQPGGSLRLSCAASGFTFSSFSMSWVRQAPGKGLEWVSSI
SGSSGTTYYADSVKGRFTISRDNSKNTLYLQMNSLRAEDTAVYYCAKPFPYFDYWGQGTLVTVSSGDGSSG
GSGGASEIVLTQSPGTLSLSPGERATLSCRASQSVSSSFLAWYQQKPGQAPRLLIYYASSRATGIPDRFSG
SGSGTDFTLTISRLEPEDFAVYYCQQTGRIPPTFGQGTKVEIKGRASGDIKLQQSGAELARPGASVKMSCK
TSGYTFTRYTMHWVKQRPGQGLEWIGYINPSRGYTNYNQKFKDKATLTTDKSSSTAYMQLSSLTSEDSAVY
YCARYYDDHYCLDYWGQGTTLTVSSVEGGSGGSGGSGGSGGVDDIQLTQSPAIMSASPGEKVTMTCRASSS
VSYMNWYQQKSGTSPKRWIYDTSKVASGVPYRFSGSGSGTSYSLTISSMEAEDAATYYCQQWSSNPLTFGA
GTKLELKHHHHHHHH (SEQ ID NO: 18)

1. A chimeric antigen receptor (CAR) comprising:
(1) an antigen-binding domain specific for the extra domain B (EDB) of fibronectin;
(2) an extracellular domain and/or a transmembrane (TM) domain of a membrane protein selected from CD3ζ, CD3ε, CD3γ, CD3δ, CD4, CD8, CD28, OX40 or CD137; and,
(3) an intracellular ITAM (Immunoreceptor Tyrosine-based Activation Motif) domain of CD3ζ, with or without a costimulatory domain;
wherein the CAR, when expressed on the surface of a T cell, is capable of activating the T cell (a) upon binding to a soluble EDB, (b) upon binding to a membrane-bound EDB, and/or (c) upon binding to EDB in extracellular matrices (e.g., those that are part of fibronectin mesh functioning as scaffold for cell attachment).

2. The CAR of claim 1, wherein the antigen-binding domain is an scFv, a single chain antibody, a nanobody (e.g., a derivative of VHH (camelid Ig)), a domain antibody (dAb, a derivative of VH or VL domain), a Bispecific T cell Engager (BiTE, a bispecific diabody); a Dual Affinity ReTargeting (DART, a bispecific diabody); an anticalin (a derivative of Lipocalins); an adnectin (10th FN3 (Fibronectin)); a Designed Ankyrin Repeat Proteins (DARPins); or an avimer.

3. The CAR of claim 1, wherein the antigen-binding domain is a human scFv or a humanized scFv.

4. The CAR of claim 1, further comprising a hinge/spacer domain between the antigen-binding domain and the TM domain.

5. The CAR of claim 4, wherein the hinge/spacer domain and the TM domain originate from the same protein.

6. The CAR of claim 5, wherein the same protein is CD8α, and wherein the hinge/spacer domain is the extracellular domain of CD8α.

7. The CAR of claim 1, wherein (3) comprises the costimulatory domain.

8. The CAR of claim 7, wherein the costimulatory domain is from CD28.

9. The CAR of claim 1, wherein (3) comprises two costimulatory domains.

10. The CAR of claim 9, wherein said two costimulatory domains comprise a costimulatory domain from CD28, and/or a costimulatory domain from CD27, 4-1BB, or OX-40.

11. The CAR of claim 1, comprising as the scFv of residues 21-236 of SEQ ID NO: 1, a CD8α extracellular and transmembrane domain, a 4-1BB intracellular domain, and a CD3ζ intracellular domain.

12. The CAR of claim 11, further comprising an N-terminal signal peptide sequence (such as the hIL-2 signal peptide sequence, or residues 1-20 of SEQ ID NO: 1).

13. The CAR of claim 12, which comprises a polypeptide of SEQ ID NO: 1.

14. A polynucleotide encoding the CAR of claim 1, such as SEQ ID NO: 2.

15. The polynucleotide of claim 14, which is codon-optimized for expression in a human cell.

16. A vector comprising the polynucleotide of claim 14.

17. The vector of claim 16, which is a viral vector capable of infecting and/or expressing said CAR in T cells, macrophages, and/or NK cells, such as primary human T cells, macrophages, or NK cells.

18. The vector of claim 17, which is a lentiviral vector.

19. The vector of claim 18, wherein the lentiviral vector is a self-inactivating lentiviral vector.

20. A cell expressing the CAR of claim 1, comprising a polynucleotide encoding the CAR of claim 1, or a vector comprising the polynucleotide.

21. The cell of claim 20, wherein the cell is an immune cell.

22. The cell of claim 20, wherein the cell is a T cell.

23. The cell of claim 20, wherein the cell is an NK cell.

24. The cell of claim 20, wherein the cell is a macrophage.

25. The cell of claim 20, wherein the cell is a primary cell isolated from a patient.

26. The cell of claim 20, wherein the cell is from an established cell line, such as an allogeneic cell line with respect to a patient to whom the cell is to be administered.

27. The cell of claim 20, wherein the cell expresses a cytokine.

28. The cell of claim 27, wherein the cytokine comprises IL-2, IL-7, IL-12, IL-15, or IL-21.

29. The cell of claim 27, wherein expression of the cytokine is under the control of a promoter that is activated by activation of the immune cell.

30. The cell of claim 20, further comprising a safety switch for down-regulating the activity of the immune cell.

31. The cell of claim 30, wherein the safety switch comprises a coding sequence for an iCaspase9 (inducible caspase-9) monomer that can be activated by dimerization with, e.g., FKBP, to trigger apoptosis of the immune cell.

32. A method of inhibiting angiogenesis in a subject having a disease or condition treatable by angiogenesis inhibition, the method comprising administering to the subject a therapeutically effective amount of an immune cell expressing a chimeric antigen receptor (CAR) comprising:
(1) an antigen-binding domain specific for the extra domain B (EDB) of fibronectin;
(2) a transmembrane (TM) domain of a membrane protein selected from CD3ζ, CD3ε, CD3γ, CD3δ, CD4, CD8, CD28, OX40 or CD137; and,
(3) an intracellular ITAM (Immunoreceptor Tyrosine-based Activation Motif) domain of CD3ζ, with or without a costimulatory domain.

33. The method of claim 32, wherein the CAR is as defined in claim 1.

34. The method of claim 32, wherein the disease or condition is a solid tumor or a chronic inflammatory condition.

35. The method of claim 34, wherein cancer cells from the solid tumor do not express EDB on cell surface.

36. The method of claim 34 or 35, wherein the disease or condition is a solid tumor, and wherein the method further comprises administering an immune checkpoint inhibitor such as a PD-1 inhibitor (e.g. pembrolizumab, nivolumab, and cemiplimab), a PD-L1 inhibitor (e.g. atezolizumab, avelumab, and durvalumab), a CTLA-4 targeting agents (e.g. ipilimumab), or an immunomodulating agent (e.g. thalidomide and lenalidomide).

37. The method of claim 34, wherein the chronic inflammatory condition is psoriasis, rheumatoid arthritis, Crohn's disease, psoriatic arthritis, ulcerative colitis, osteoarthritis, asthma, pulmonary fibrosis, IBD, inflammation-induced lymphangiogenesis, obesity, diabetes, retinal neovascularization (RNV), diabetic retinopathy, choroidal neovascularization (CNV), age-related macular degeneration (AMD), metabolic syndrome-associated disorder, prolonged peritoneal dialysis, juvenile arthritis, or atherosclerosis.

38. The method of claim 32 further comprising administering a second therapeutic agent effective to inhibit angiogenesis.

39. The method of claim 38, wherein the second therapeutic agent comprises axitinib, bevacizumab, cabozantinib, everolimus, lenalidomide, pazopanib, ramucirumab, regorafenib, sorafenib, sunitinib, thalidomide, vandetanib, and/or ziv-aflibercept.

40. The method of claim 32, wherein the immune cell is produced by introducing in vitro a vector of claim 16 into a primary immune cell isolated from the subject, and optionally culturing and/or expanding in vitro the primary immune cells introduced by the vector.

41. The method of claim 32, further comprising administering a reagent that suppresses cytokine release syndrome (CRS), such as an anti-IL-6 monoclonal antibody (e.g., tocilizumab); and/or immunoglobulin therapy.
